(19) United States
(12) Patent Application Publication
(10) Pub. No.: US 2006/0165620 A1
(43) Pub. Date: Jul. 27, 2006

(54) Coloured gloss pigments having at least one coating of siox, with x=0.03 to 0.95 for use in cosmetic and personal care formulations

(76) Inventors: Patrice Bujard, Reinach (CH); Oliver Reich, Grenzach-Wyhlen (DE)

Correspondence Address: CIBA SPECIALTY CHEMICALS CORPORATION;PATENT DEPARTMENT, 540 WHITE PLAINS RD, P O BOX 2005, TARRYTOWN, NY, 10591-9005, US

(21) Appl. No.: 10/524,457

(22) PCT Filed: Aug. 21, 2003
(86) PCT No.: PCT/EP03/09269

§ 371 Date: Feb. 10, 2005

(30) Foreign Application Priority Data
Aug. 30, 2002 (EP) 02405749.9
Jan. 22, 2003 (EP) 03405024.5

(51) Int. Cl. A61K 8/29; A61K 8/25; C04B 14/04

(52) U.S. Cl. 424/63; 106/482

(57) ABSTRACT

The present invention relates to a cosmetic and personal care preparation or formulation comprising (a) from 0.0001 to 90% by weight of a gloss pigment comprising (a1) a core consisting of a substantially transparent or metallically reflecting material, and (a2) at least one coating substantially consisting of one or more silicon oxides, the molar ratio of oxygen to silicon being on average from 0.03 to 0.95 and (b) from 10 to 99. 9999% of a (cosmetically) suitable carrier material, based on the total weight of the cosmetic preparation or formulation.

The present invention relates to a cosmetic and personal care preparation or formulation comprising

(a) from 0.0001 to 90% by weight of a gloss pigment comprising
(a1) a core consisting of a substantially transparent or metallically reflecting material, and
(a2) at least one coating substantially consisting of one or more silicon oxides, the molar ratio of oxygen to silicon being on average from 0.03 to 0.95 and

(b) from 10 to 99.9999% of a (cosmetically) suitable carrier material, based on the total weight of the cosmetic preparation or formulation.

Makeup compositions, such as free powders, compact powders, foundations, blushes, eyeshadows, and lipsticks, may comprise an appropriate vehicle and various coloring agents intended to confer a certain color on the compositions, before and/or after their application to at least one of the-skin (including the lips), and the superficial body growths.

A fairly restricted range of coloring agents, for example pigments, such as lakes, inorganic pigments, and pearlescent pigments, is currently used to create colors. Lakes make it possible to obtain vivid colors but are generally unstable to light, temperature, and pH. After application, some also stain the skin in an unsightly way, by discharge of the colorant. Inorganic pigments, for example inorganic oxides, are, in contrast, very stable but give rather dull and pale colors. Pearlescent pigments do not provide intense colors with an iridescent effect. Rather, pearlescent pigments make it possible to obtain varied colors with generally fairly weak color effects, for example, the color effect is mainly visible along only one given angle corresponding to a spectral reflection. For example, patent WO-A-96/03962 discloses a cosmetic composition in the form of an emulsion comprising a silicone oil, an “interferential” pigment of platelet type composed of a support such as mica coated with a given layer of titanium oxide with a given thickness, and a pigment based on iron oxide. Due to the interferential pigment present in the composition, the composition gives a shade along a given angle and does not produce a goniochromatic effect.

One of the objects of the present invention is to provide a cosmetic composition which overcomes at least one of the above-mentioned disadvantages. Another of the objects of the present invention is to provide a cosmetic composition which exhibits at least one of the following effects, for example aesthetic, goniochromatic, and volume effects. Such effects may be obtained by use of compositions, for example, chosen from foundations, eye shadows, blushes, lipsticks, lip glosses, lip lacquers, mascaras, and eyeliners.

Accordingly, the present invention relates to a cosmetic and personal care preparation or formulation comprising

(a) from 0.0001 to 90% by weight of a gloss pigment comprising
(a1) a core consisting of a substantially transparent or metallically reflecting material, and
(a2) at least one coating substantially consisting of one or more silicon oxides, the molar ratio of oxygen to silicon being on average from 0.03 to 0.95 and

(b) from 10 to 99.9999% of a (cosmetically) suitable carrier material, based on the total weight of the cosmetic preparation or formulation.

The preparations according to the invention are especially preparations or formulations that are suitable for making-up the lips or the skin and for colouring the hair or the nails.

The cosmetic preparations are, for example, lipsticks, blushers, foundations, nail varnishes and hair shampoos.

The gloss pigments may be used singly or in the form of mixtures. It is, in addition, possible to use the gloss pigments together with other pigments and/or colorants.

The cosmetic preparations and formulations according to the invention preferably contain the gloss pigment in an amount from 0.005 to 50% by weight, based on the total weight of the preparation.

Suitable carrier materials for the cosmetic preparations and formulations according to the invention include the customary materials used in such compositions.

The cosmetic preparations and formulations according to the invention may be in the form of, for example, sticks, ointments, creams, emulsions, suspensions, dispersions, powders or solutions. They are, for example, lipsticks, mascara preparations, blushes, eye-shadows, foundations, eyeliners, powder or nail varnishes.

If the preparations are in the form of sticks, for example lipsticks, eye-shadows, blushes or foundations, the preparations consist for a considerable part of fatty components, which may consist of one or more waxes, for example ozokerite, lanolin, lanolin alcohol, hydrogenated lanolin, acetylated lanolin, lanolin wax, beeswax, candelilla wax, microcrystalline wax, carnauba wax, cetyl alcohol, stearyl alcohol, cocoa butter, lanolin fatty acids, petrolatum, petroleum jelly, mono-, di- or tri-glycerides or fatty esters thereof that are solid at 25° C., silicone waxes, such as methyloctadecane-oxypolysiloxane and poly(dimethylsiloxy)-stearoxysiloxane, stearic acid monoethanolamine, colophane and derivatives thereof, such as glycol abietates and glycerol abietates, hydrogenated oils that are solid at 25° C., sugar glycerides and oleates, myristates, lanolates, stearates and dihydroxystearates of calcium, magnesium, zirconium and aluminium.

The fatty component may also consist of a mixture of at least one wax and at least one oil, in which case the following oils, for example, are suitable: paraffin oil, purcelline oil, perhydrosqualene, sweet almond oil, avocado oil, calophyllum oil, castor oil, sesame oil, jojoba oil, mineral oils having a boiling point about from 310 to 41 0C, silicone oils, such as dimethylpolysiloxane, linoleyl alcohol, linolenyl alcohol, oleyl alcohol, cereal grain oils, such as wheatgerm oil, isopropyl lanolate, isopropyl palmitate, isopropyl myristate, butyl myristate, cetyl myristate, hexadecyl stearate, butyl stearate, decyl oleate, acetyl glycerides, octanoates and decanoates of alcohols and polyalcohols, for example of glycol and glycerol, ricinoleates of alcohols and polyalcohols, for example of cetyl alcohol, isostearyl alcohol, isocetyl lanolate, isopropyl adipate, hexyl laurate and octyl dodecanol.

The fatty components in such preparations in the form of sticks may generally constitute up to 99.91% by weight of the total weight of the preparation.

The cosmetic preparations and formulations according to the invention may additionally comprise further constituents, such as, for example, glycols, polyethylene glycols, polypropylene glycols, monoalkanolamides, non-coloured polymeric, inorganic or organic fillers, preservatives, UV filters or other adjuvants and-additives customary in cosmetics, for example a natural or synthetic or partially synthetic di- or tri-glyceride, a mineral oil, a silicone oil, a wax, a fatty alcohol, a Guerbet alcohol or ester thereof, a lipophilic functional cosmetic active ingredient, including sun-protection filters, or a mixture-of such substances.

A lipophilic functional cosmetic active ingredient suitable for skin cosmetics, an active ingredient composition or an active ingredient extract is an ingredient or a mixture of ingredients that is approved for dermal or topical application. The following may be mentioned by way of example:
active ingredients having a cleansing action on the skin surface and the hair; these include all substances that serve to cleanse the skin, such as oils, soaps, synthetic detergents and solid substances;
active ingredients having a deodorising and perspiration-inhibiting action: they include antiperspirants based on aluminium salts or zinc salts, deodorants comprising bactericidal or bacteriostatc deodorising substances, for example triclosan, hexachlorophene, alcohols and cationic substances, such as, for example, quaternary ammonium salts, and odour absorbers, for example ®Grillocin (combination of zinc ricinoleate and various additives) or triethyl citrate (optionally in combination with an antioxidant, such as, for example, butyl hydroxytoluene) or ion-exchange resins; active ingredients that offer protection against sunlight (UV filters): suitable active ingredients are filter substances (sunscreens) that are able to absorb UV radiation from sunlight and convert it into heat; depending on the desired action, the following light-protection agents are preferred: light-protection agents that selectively absorb sunburn-causing high-energy UV radiation in the range of approximately from 280 to 315 nm (UV-B absorbers) and transmit the longer-wavelength range of, for example, from 315 to 400 nm (UV-A range), as well as light-protection agents that absorb only the longer-wavelength radiation of the UV-A range of from 315 to 400 nm (UV-A absorbers);

suitable light-protection agents are, for example, organic UV absorbers from the class of the p-aminobenzoic acid derivatives, salicylic acid derivatives, benzophenone derivatives, dibenzoylmethane derivatives, diphenyl acrylate derivatives, benzofuran derivatives, polymeric UV absorbers comprising one or more organosilicon radicals, cinnamic acid derivatives, camphor derivatives, trianilino-s-triazine derivatives, phenyl-benzimidazolesulfonic acid and salts thereof, menthyl anthranilates, benzotriazole derivatives, and/or an inorganic micropigment selected from aluminium oxide- or silicon dioxide-coated TiO₂, zinc oxide or mica; wherein suitable UV filter substances are listed below:

| Chemical Name | CAS No. |
|---|---|
| (+/−)-1,7,7-trimethyl-3-[(4-methylphenyl)methylene]bicyclo[2.2.1]heptan-2- | 36861-47-9 |
| one | |
| 1,7,7-trimethyl-3-(phenylmethylene)bicyclo[2.2.1]heptan-2-one | 15087-24-8 |
| (2-Hydroxy-4-methoxyphenyl)(4-methylphenyl)methanone | 1641-17-4 |
| 2,4-dihydroxybenzophenone | 131-56-6 |
| 2,2′,4,4′-tetrahydroxybenzophenone | 131-55-5 |
| 2-Hydroxy-4-methoxy benzophenone; | 131-57-7 |
| 2-Hydroxy-4-methoxy benzophenone-5-sulfonic acid | 4065-45-6 |
| 2,2′-dihydroxy-4,4′-dimethoxybenzophenone | 131-54-4 |
| 2,2′-Dihydroxy-4-methoxybenzophenone | 131-53-3 |
| Alpha-(2-oxoborn-3-ylidene)toluene-4-sulphonic acid and its salts | 56039-58-8 |
| 1-[4-(1,1-dimethylethyl)phenyl]-3-(4-methoxyphenyl)propane-1,3-dione | 70356-09-1 |
| Methyl N,N,N-trimethyl-4-[(4,7,7-trimethyl-3-oxobicyclo[2,2,1]hept-2- | 52793-97-2 |
| ylidene)methyl]anilinium sulphate; | |
| 3,3,5-Trimethyl cyclohexyl-2-hydroxy benzoate | 118-56-9 |
| Isopentyl p-methoxycinnamate | 71617-10-2 |
| Menthyl-o-aminobenzoate | 134-09-8 |
| Menthyl salicylate | 89-46-3 |
| 2-Ethylhexyl 2-cyano, 3,3-diphenylacrylate | 6197-30-4 |
| 2-ethylhexyl 4-(dimethylamino)benzoate | 21245-02-3 |
| 2-ethylhexyl 4-methoxycinnamate | 5466-77-3 |
| 2-ethylhexyl salicylate | 118-60-5 |
| Benzoic acid, 4,4′,4″-(1,3,5-triazine-2,4,6-triyltriimino)tris-, tris(2- | 88122-99-0 |
| ethylhexyl)ester; 2,4,6-Trianilino-(p-carbo-2′-ethylhexyl-1′-oxi)-1,3,5- | |
| triazine | |
| 4-aminobenzoic acid | 150-13-0 |
| Benzoic acid, 4-amino-, ethyl ester, polymer with oxirane | 113010-52-9 |
| 2-phenyl-1H-benzimidazole-5-sulphonic acid | 27503-81-7 |
| 2-Propenamide, N-[[4-[(4,7,7-trimethyl-3-oxobicyclo[2.2.1]hept-2- | 147897-12-9 |
| ylidene)methyl]phenyl]methyl]-, homopolymer | |
| Triethanolamine salicylate | 2174-16-5 |
| 3,3′-(1,4-phenylenedimethylene)bis[7,7-dimethyl-2-oxo- | 90457-82-2 |
| bicyclo[2.2.1]heptane-1-methanesulfonic acid] | |
| Titanium dioxide | 13463-67-7 |
| Zinc oxide | 1314-13-2 |
| 2,2′-Methylene-bis-[6-(2H-benzotriazol-2-yl)-4-(1,1,3,3-tetramethylbutyl)- | 103597-45-1 |
| phenol] | |
| 2,4-bis{[4-(2-ethylhexyloxy)-2-hydroxy]-phenyl}-6-(4-methoxyphenyl)- | 187393-00-6 |
| (1,3,5)-triazine | |
| 1H-Benzimidazole-4,6-disulfonic acid, 2,2′-(1,4-phenylene)bis-, disodium | 180898-37-7 |
| salt | |
| Benzoic acid, 4,4′-[[6-[[4-[[(1,1- | 154702-15-5 |
| dimethylethyl)amino]carbonyl]phenyl]amino]1,3,5-triazine-2,4- | |
| diyl]diimino]bis-, bis(2-ethylhexyl)ester | |
| Phenol, 2-(2H-benzotriazol-2-yl)-4-methyl-6-[2-methyl-3-[1,3,3,3- | 155633-54-8 |
| tetramethyl-1-[(trimethylsilyl)oxy]disiloxanyl]propyl]- | |
| Dimethicodiethylbezalmalonate | 207574-74-1 |
| Benzenesulfonic acid, 3-(2H-benzotriazol-2-yl)-4-hydroxy-5-(1-methyl- | 92484-48-5 |
| propyl)-, monosodium salt | |
| Benzoic acid, 2-[4-(diethylamino)-2-hydroxybenzoyl]-, hexyl ester | 302776-68-7 |
| 1-Dodecanaminium, N-[3-[[4-(dimethylamino)benzoyl]amino]propyl]N,N- | 156679-41-3 |
| dimethyl-, salt with 4-methylbenzenesulfonic acid (1:1) | |
| 1-Propanaminium, N,N,N-trimethyl-3-[(1-oxo-3-phenyl-2-propenyl)amino]-, | 177190-98-6 |
| chloride | |
| 1H-Benzimidazole-4,6-disulfonic acid, 2,2′-(1,4-phenylene)bis- | 170864-82-1 |
| 1,3,5-Triazine, 2,4,6-tris(4-methoxyphenyl)- | 7753-12-0 |
| 1,3,5-Triazine, 2,4,6-tris[4-[(2-ethylhexyl)oxy]phenyl]- | 208114-14-1 |
| 1-Propanaminium, 3-[[3-[3-(2H-benzotriazol-2-yl)-5-(1,1-dimethylethyl)-4- | 340964-15-0 |
| hydroxyphenyl]-1-oxopropyl]amino]-N,N-diethyl-N-methyl-, methyl sulfate | |
| (salt) | |
| 2-Propenoic acid, 3-(1H-imidazol-4-yl)- | 104-98-3 |
| Benzoic acid, 2-hydroxy-, [4-(1-methylethyl)phenyl]methyl ester | 94134-93-7 |
| 1,2,3-Propanetriol, 1-(4-aminobenzoate) | 136-44-7 |
| Benzeneacetic acid, 3,4-dimethoxy-a-oxo- | 4732-70-1 |
| 2-Propenoic acid, 2-cyano-3,3-diphenyl-, ethyl ester | 5232-99-5 |
| Anthralinic acid, p-menth-3-yl ester | 134-09-8 |
| 2,2′-bis(1,4-phenylene)-1H-benzimidazole-4,6-disulphonic acid mono | 349580-12-7 |
| sodium salt or Disodium phenyl dibenzimidazole tetrasulfonate or Heliopan | |
| AP | |

active ingredients against insects (repellents) are agents that are intended to prevent insects from touching the skin and becoming active there; they drive insects away and evaporate slowly; the most frequently used repellent is diethyl toluamide (DEET); other common repellents will be found in “Pflegekosmetik” (W. Raab and U. Kindl, Gustav-Fischer-Verlag Stuttgart/New York, 1991) on page 161;
active ingredients for protection against chemical and mechanical influences: these include all substances that form a barrier between the skin and external harmful substances, such as, for example, paraffin oils, silicone oils, vegetable oils, PCL products and lanolin for protection against aqueous solutions, film-forming agents, such as sodium alginate, triethanolamine alginate, polyacrylates, polyvinyl alcohol or cellulose ethers for protection against the effect of organic solvents, or substances based on mineral oils, vegetable oils or silicone oils as “lubricantst” for protection against severe mechanical stresses on the skin;
moisturising substances: the following substances, for example, are used as moisture-controlling agents (moisturisers): sodium lactate, urea, alcohols, sorbitol, glycerol, propylene glycol, collagen, elastin and hyaluronic acid;
active ingredients having a keratoplastic effect: benzoyl peroxide, retinoic acid, colloidal sulfur and resorcinol;
antimicrobial agents, such as, for example, triclosan or quaternary ammonium compounds;
oily or oil-soluble vitamins or vitamin derivatives that can be applied dermally: for example vitamin A (retinol in the form of the free acid or derivatives thereof), panthenol, pantothenic acid, folic acid, and combinations thereof, vitamin E (tocopherol), vitamin F; essential fatty acids; or niacinamide (nicotinic acid amide);
vitamin-based placenta extracts: active ingredient compositions comprising especially vitamins A, C, E, B₁, B₂, B₆, B₁₂, folic acid and biotin, amino acids and enzymes as well as compounds of the trace elements magnesium, silicon, phosphorus, calcium, manganese, iron or copper;
skin repair complexes: obtainable from inactivated and disintegrated cultures of bacteria of the bifidus group;
plants and plant extracts: for example arnica, aloe, beard lichen, ivy, stinging nettle, ginseng, henna, camomile, marigold, rosemary, sage, horsetail or thyme;
animal extracts: for example royal jelly, propolis, proteins or thymus extracts;
cosmetic oils that can be applied dermally: nonionic glycerol esters, for example Miglyol 812, apricot kernel oil, avocado oil, babassu oil, cottonseed oil, borage oil, thistle oil, groundnut oil, gamma-oryzanol, rosehip-seed oil, hemp oil, hazelnut oil, blackcurrant-seed oil, jojoba oil, cherry-stone oil, salmon oil, linseed oil, cornseed oil, macadamia nut oil, almond oil, evening primrose oil, mink oil, olive oil, pecan nut oil, peach kernel oil, pistachio nut oil, rape oil, rice-seed oil, castor oil, safflower oil, sesame oil, soybean oil, sunflower oil, tea tree oil, grapeseed oil or wheatgerm oil.

The preparations in stick form are preferably anhydrous but may in certain cases comprise a certain amount of water which, however, in general does not exceed 40% by weight, based on the total weight of the cosmetic preparation.

If the cosmetic preparations and formulations according to the invention are in the form of semi-solid products, that is-to say in the form of ointments or creams, they may likewise be anhydrous or aqueous. Such preparations and formulations are, for example, mascaras, eyeliners, foundations, blushers, eye-shadows, or compositions for treating rings under the eyes.

If, on the other hand, such ointments or creams are aqueous, they are especially emulsions of the water-in-oil type or of the oil-in-water type that comprise, apart from the pigment, from 1 to 98.8% by weight of the fatty phase, from 1 to 98.8% by weight of the aqueous phase and from 0.2 to 30% by weight of an emulsifier.

Such ointments and creams may also comprise further conventional additives, such as, for example, perfumes, antioxidants, preservatives, gel-forming agents, UV filters, colorants, pigments, pearlescent agents, non-coloured polymers as well as inorganic or organic fillers. If the preparations are in the form of a powder, they consist substantially of a mineral or inorganic or organic filler such as, for example, talcum, kaolin, starch, polyethylene powder or polyamide powder, as well as adjuvants such as binders, colorants etc.

Such preparations may likewise comprise various adjuvants conventionally employed in cosmetics, such as fragrances, antioxidants, preservatives etc.

If the cosmetic preparations and formulations according to the invention are nail varnishes, they consist essentially of nitrocellulose and a natural or synthetic polymer in the form of a solution in a solvent system, it being possible for the solution to comprise other adjuvants, for example pearlescent agents. In that embodiment, the coloured polymer is present in an amount of approximately from 0.1 to 5% by weight.

The cosmetic preparations and formulations according to the invention may also be used for colouring the hair, in which case they are used in the form of shampoos, creams or gels that are composed of the base substances conventionally employed in the cosmetics industry and a pigment according to the invention.

The compositions for colouring the hair may furthermore comprise any active ingredient, additive or adjuvant known” for such preparations.

Adjuvants that are suitable for such formulations are in general customary in the field hair-colouring, such as for example surfactants or tensides, solvents, bases, acids, perfumes, polymeric adjuvant, thickeners and light stabilisers.

The composition for colouring the hair in many cases comprises at least one surfactant. Suitable surfactants are anionic, zwitterionic, ampholytic, non-ionic and cationic surfactants. In many cases, however, it has proved advantageous to select the surfactants from anionic, zwitterionic and non-ionic surfactants.

Anionic surfactants suitable for use in the compositions for colouring the hair include all anionic surface-active substances that are suitable for use on the human body. Such substances are characterised by an anionic group that imparts water solubility, for example a carboxylate, sulfate, sulfonate or phosphate group, and a lipophilic alkyl group having approximately from 10 to 22 carbon atoms. In addition, glycol or polyglycol ether groups, ester, ether and amide groups and also hydroxy groups may be present in the molecule. The following are examples of suitable anionic surfactants, each in the form of sodium, potassium or ammonium salts or mono-, di- or tri-alkanolammonium salts having 2 or 3 carbon atoms in the alkanol group:
linear fatty acids having from 10 to 22 carbon atoms (soaps),
ether carboxylic acids of formula R—O—(CH₂—CH₂—O)ₓ—CH₂—COOH, in which R is a linear alkyl group having from 10 to 22 carbon atoms and x=0 or from 1 to 16,
acyl sarcosides having from 10 to 18 carbon atoms in the acyl group,
acyl tau rides having from 10 to 18 carbon atoms in the acyl group,
acyl isothionates having from 10 to 18 carbon atoms in the acyl group,
sulfosuccinic mono- and di-alkyl esters having from 8 to 18 carbon atoms in the alkyl group and sulfosuccinic monoalkylpolyoxyethyl esters having from 8 to 18 carbon atoms in the alkyl group and from 1 to 6 oxyethyl groups,
linear alkane sulfonates having from 12 to 18 carbon atoms,
linear α-olefin sulfonates having from 12 to 18 carbon atoms,
α-sulfo fatty acid methyl esters of fatty acids having from 12 to 18 carbon atoms,
alkyl sulfates and alkyl polyglycol ether sulfates of formula R′—O(CH2-CH2-O)x′-SO3H, in which R′ is a preferably linear alkyl group having from 10 to 18 carbon atoms and x′=0 or from 1 to 12,
mixtures of surface-active hydroxysulfonates according to DE-A-3 725 030, especially page 3, lines 40 to 55,
sulfated hydroxyalkylpolyethylene and/or hydroxyalkylenepropylene glycol ethers according to DE-A-3 723 354, especially page 4, lines 42 to 62,
sulfonates of unsaturated fatty acids having from 12 to 24 carbon atoms and from 1 to 6 double bonds according to DE-A-3 926 344, especially page 2, lines 36 to 54,
esters of tartaric acid and citric acid with alcohols which are addition products of approximately from 2 to 15 molecules of ethylene oxide and/or propylene oxide with fatty alcohols having from 8 to 22 carbon atoms, or
anionic surfactants, as described in WO 00/10518, especially page 45, line 11 to page 48, line 3.

Preferred anionic surfactants are alkyl sulfates, alkyl polyglycol ether sulfates and ether carboxylic acids having from 10 to 18 carbon atoms in the alkyl group and up to 12 glycol ether groups in the molecule, and also especially salts of saturated and especially unsaturated C₈-C₂₂carboxylic acids, such as oleic acid, stearic acid, isostearic acid and palmitic acid.

Surface-active compounds that carry at least one quaternary ammonium group and at least one —COO⁽⁻⁾ or —SO₃⁽⁻⁾ group in the molecule are termed zwitterionic surfactants. Zwitterionic surfactants that are especially suitable are the so-called betaines, such as the N-alkyl-N,N-dimethylammonium glycinates, for example cocoalkyldimethylammonium glycinate,

N-acylaminopropyl-N,N-dimethylammonium glycinates, for example cocoacylaminopropyl-dimethylammonium glycinate, and 2-alkyl-3-carboxymethyl-3-hydroxyethylimidazolines having from 8 to 18 carbon atoms in the alkyl or acyl group and also cocoacylaminoethylhydroxyethylcarboxymethyl glycinate. A preferred zwitterionic surfactant is the fatty acid amide derivative known by the CTFA name cocoamidopropyl betaine. Ampholytic surfactants are to be understood as meaning surface-active compounds that, in addition to a C₈-C₁₈-alkyl or -acyl group, contain at least one free amino group and at least one —COOH or —SO₃H group in the molecule and are capable of forming internal salts. Examples of suitable ampholytic surfactants include N-alkylglycines, N-alkylpropionic acids, N-alkylaminobutyric acids, N-alkyliminodipropionic acids, N-hydroxyethyl-N-alkylamidopropyl-glycines, N-alkyltaurines, N-alkylsarcosines, 2-alkylaminopropionic acids and alkylaminoacetic acids, each having approximately from 8 to 18 carbon atoms in the alkyl group. Ampholytic surfactants to which special preference is given are N-cocoalkyl-aminopropionate, cocoacylaminoethylaminopropionate and C₁₂-C₁₈acylsarcosine. Non-ionic surfactants are described in WO 00/1 0519, especially page 45, line 11 to page 50, line 12.

Non-ionic surfactants contain as the hydrophilic group, for example, a polyol group, a polyalkylene glycol ether group or a combination of polyol and polyglycol ether groups. Such compounds are, for example:
addition products of from 2 to 30 mol of ethylene oxide and/or from 0 to 5 mol of propylene oxide with linear fatty alcohols having from 8 to 22 carbon atoms, with fatty acids having from 12 to 22 carbon atoms and with alkylphenols having from 8 to 15 carbon atoms in the alkyl group,
C₁₂-C₂₂ fatty acid mono- and di-esters of addition products of from 1 to 30 mol of ethylene oxide with glycerol,
C₈-C₂₂alkyl-mono- and -oligo-glycosides and ethoxylated analogues thereof,
addition products of from 5 to 60 mol of ethylene oxide with castor oil and hydrogenated castor oil,
addition products of ethylene oxide with sorbitan fatty acid esters,
addition products of ethylene oxide with fatty acid alkanolamides.

Examples of cationic surfactants that can be used in the preparations (compositions) according to the invention are especially quaternary ammonium compounds. Preference is given to ammonium halides, such as alkyltrimethylammonium chlorides, dialkyldimethylammonium chlorides and trialkylmethylammonium chlorides, for example cetyltrimethylammonium chloride, stearyltrimethylammonium chloride, distearyldimethy-lammonium chloride, lauryidimethylammonium chloride, lauryldimethylbenzylammonium chloride and tricetylmethylammonium chloride. Further cationic surfactants that can be used in accordance with the invention are quaternised protein hydrolysates.

Also suitable in accordance with the invention are cationic silicone oils, such as, for example, the commercially available products Q2-7224 (manufacturer: Dow Corning; a stabilised trimethylsilylamodimethicone), Dow Corning 929 emulsion (comprising a hydroxylamino-modified silicone, which is also referred to as amodimethicone), SM-2059 (manufacturer:

General Electric), SLM-55067 (manufacturer: Wacker) and also Abil®-Quat 3270 and 3272 (manufacturer: Th. Goldschmidt; diquaternary polydimethylsiloxanes, quaternium-80), or silicones, as described in WO 00/12057, especially page 45, line 9 to page 55, line 2. Alkylamidoamines, especially fatty acid amidoamines, such as the stearylamidopropyl-dimethylamine obtainable under the name Tego Amid® 18, are distinguished not only by a good conditioning action but also especially by their good biodegradability.

Quaternary ester compounds, so-called “esterquats”, such as the methyl hydroxyalkyl-dialkoyloxyalkylammonium methosulfates marketed under the trademark Stepantex), are also very readily biodegradable.

An example of a quaternary sugar derivative that can be used as cationic surfactant is the commercial product Glucquat® 100, according to CTFA nomenclature a “lauryl methyl gluceth-10 hydroxypropyl dimonium chloride”.

The alkyl-group-containing compounds used as surfactants may be single substances, but the use of natural raw materials of vegetable or animal origin is generally preferred in the preparation of such substances, with the result that the substance mixtures obtained have different alkyl chain lengths according to the particular starting material used.

The surfactants that are addition products of ethylene and/or propylene oxide with fatty alcohols or derivatives of such addition products may either be products having a “normal” homologue distribution or products having a restricted homologue distribution. “Normal” homologue distribution is to be understood as meaning mixtures of homologues obtained in the reaction of fatty alcohol and alkylene oxide using alkali metals, alkali metal hydroxides or alkali metal alcoholates as catalysts. Restricted homologue distributions, on the other hand, are obtained when, for example, hydrotalcites, alkali metal salts of ether carboxylic acids, alkali metal oxides, hydroxides or alcoholates are used as catalysts. The use of products having restricted homologue distribution may be preferred.

Further preferred active ingredients of formulation according to the present invention, adjuvants and additives are as follows:
non-ionic polymers, for example vinylpyrrolidonelvinyl acrylate copolymers, polyvinyl-pyrrolidone and vinylpyrrolidone/vinyl acetate copolymers and polysiloxanes,
cationic polymers, such as quaternised cellulose ethers, polysiloxanes having quaternary groups, dimethyidiallylammonium chloride polymers, copolymers of dimethyldiallyl-ammonium chloride and acrylic acid, as available commercially under the name Merquat® 280 and the use of which in hair colouring is described, for example, in DE-A-4 421 031, especially page 2, lines 20 to 49, or EP-A-953 334, especially page 27, line 17 to page 30, line 11, acrylamideldimethyldiallylammonium chloride copolymers, diethyl-sulfate-quaternised dimethylaminoethyl methacrylate/vinylpyrrolidone copolymers, vinylpyrrolidone/imidazolinium methochloride copolymers,
quaternised polyvinyl alcohol,
zwitterionic and amphoteric polymers, such as, for example, acrylamido-propyl-trimethylammonium chloride/acrylate copolymers and octylacrylamide/methyl methacrylate/tert-butylaminoethyl methacrylate/2-hydroxypropyl methacrylate copolymers,
anionic polymers, such as, for example, polyacrylic acids, crosslinked polyacrylic acids, vinyl acetate/crotonic acid copolymers, vinylpyrrolidone/vinyl acrylate copolymers, vinyl acetate/butyl maleate/isobornyl acrylate copolymers, methyl vinyl ether/maleic anhydride copolymers and acrylic acid/ethyl acrylate/N-tert-butyl acrylamide terpolymers,
thickeners, such as agar, guar gum, alginates, xanthan gum, gum arabic, karaya gum, locust bean flour, linseed gums, dextrans, cellulose derivatives, e.g. methyl cellulose, hydroxyalkyl cellulose and carboxymethyl cellulose, starch fractions and derivatives, such amylose, amylopectin and dextrins, clays, e.g. bentonite or fully synthetic hydrocolloids such as, for example, polyvinyl alcohol, or Salcare range such as Salcare SC80(steareth-10 allyl ether/acrylates copolymer), Salcare SC81 (acrylates copolymer), Salcare SC91 and Salcare AST(sodium acrylates copolymer/PPG-1 trideceth-6),
structuring agents, such as glucose and maleic acid,
hair-conditioning compounds, such as phospholipids, for example soya lecithin, egg lecithin, and cephalins, silicone oils, and also conditioning compounds, for example such as those described in DE-A-1 9 729 080, especially page 2, lines 20 to 49, EP-A-834 303, especially page 2, line 18 to page 3, line 2, or EP-A-312 343, especially page 2, line 59 to page 3, line 11,
protein hydrolysates, especially elastin, collagen, keratin, milk protein, soya protein and wheat protein hydrolysates, condensation products thereof with fatty acids and also quaternised protein hydrolysates,
perfume oils, dimethyl isosorbitol and cyclodextrins,
solubilisers, such as ethanol, isopropanol, ethylene glycol, propylene glycol, glycerol and diethylene glycol,
anti-dandruff active ingredients, such as piroctones, olamines and zinc Omadine,
further substances for adjusting the pH value,
active ingredients such as panthenol, pantothenic acid, allantoin, pyrrolidonecarboxylic acids and salts thereof, plant extracts and vitamins,
cholesterol,
light stabilisers and UV absorbers, as described, for example, in EP-A-819 422, especially page 4, lines 34 to 37,
consistency regulators, such as sugar esters, polyol esters or polyol alkyl ethers,
fats and waxes, such as spermaceti, beeswax, montan wax, paraffins, fatty alcohols and fatty acid esters,
fatty alkanolamides,
polyethylene glycols and polypropylene glycols having a molecular weight of from 150 to 50 000, for example such as those described in EP-A-801 942, especially page 3, lines 44 to 55,
complexing agents, such as EDTA, NTA and phosphonic acids,
swelling and penetration substances, such as polyols and polyol ethers, as listed extensively, for example, in EP-A-962 219, especially page 27, lines 18 to 38, for example glycerol, propylene glycol, propylene glycol monoethyl ether, butyl glycol, benzyl alcohol, carbonates, hydrogen carbonates, guanidines, ureas and also primary, secondary and tertiary phosphates, imidazoles, tannins, pyrrole,
opacifiers, such as latex,
pearlising agents, such as ethylene glycol mono- and di-stearate,
propellants, such as propane-butane mixtures, N₂O, dimethyl ether, CO₂ and air, and also
antioxidants,
polyols or polyethers, as described in EP-A-962 219, especially page 27, lines 14 to 38,
thickening polymers, as described in EP-A-970 684, especially page 48, line 16 to page 51, line 4,
sugar-containing polymers, as described in EP-A-970 687, especially page 28, line 17 to page 29, line 23,
quaternary ammonium salts, as described in WO 00/1 0517, especially page 44, line 16 to page 46, line 23.

The use of UV absorbers can effectively protect natural and dyed hair from the damaging rays of the sun and increase the wash fastness of dyed hair.

Preferred UV absorbers in the compositions for colouring the hair are:
cationic benzotriazole UV absorbers as for example described in WO 01/36396 especially on page 1, line 20 to page 2, line 24, and preferred on page 3 to 5, and on pages 26 to 37, or
cationic benzotriazole UV in combination with antioxidants as described in WO 01/36396, especially on page 11, line 14 to page 18, or
UV absorbers in combination with antioxidants as described in U.S. Pat. No. 5,922,310, especially in column 2, lines 1 to 3,
UV absorbers in combination with antioxidants as described in U.S. Pat. No. 4,786,493, especially in column 1, 42 to column 2, line 7, and preferred in column 3, 43 to column 5, line 20, or
combination of UV absorbers as described in U.S. Pat. No. 5,830,441, especially in column 4, lines 53 to 56, or
combination of UV absorbers as described in WO 01/36396, especially on page 11, lines 9 to 13, or
triazine derivatives provide effective UV protection as described in WO 98/22447, especially on page 1, line 23 to page 2, line 4, and preferred on page 2, line 11 to page 3, line 15 and most preferred on pages 6 to 7, and 12 to 16, or

combination of the cosmetic formulations as described in WO 98/22447 with one or more than one further UV filter as described in the following patents:

| | |
|---|---|
| EP 895776 | Comp. in Rows 48-58, p 3; R 25 + 33, p 5 |
| WO 9220690 | Polymeric comp in Examples 3-6 |
| EP 1000950 | Comp. in Table 1, pp 18-21 |
| EP 1060734 | T 1-3, pp 11-14 |
| EP 1059082 | Ex 1; T 1, pp 9-11 |
| EP 1008586 | Ex 1-3, pp 13-15 |
| EP 1005855 | T 3, p 13 |
| EP 1129695 | Ex 1-7, pp 13-14 |
| EP 967200 | Ex 2, T 3-5, pp 17-20 |
| EP 945125 | T 3 a + b, pp 14-15 |
| EP 924246 | T 2, p 9 |
| EP 911020 | T 2, p 11-12 |
| EP 916335 | T 2-4, pp 19-41 |
| EP 852137 | T 2, pp 41-46 |
| EP 858318 | T 1, p 6 |
| EP 826361 | T 1, pp 5-6 |
| EP 503338 | T 1, pp 9-10 |
| WO 9301164 | T 1 + 2, pp 13-22 |
| EP 823418 | Ex 1-4, pp 7-8 |
| WO 9714680 | Ex 1-3, p 10 |
| EP 1027883 | Compound VII, p 3 |
| EP 832641 | Ex 5 + 6 p 7; t 2, p 8 |
| U.S. Pat. No. 5338539 | Ex 1-9, pp 3 + 4 |
| EP 517103 | Ex 3, 4, 9, 10 pp 6-7 |
| EP 1123934 | T 3, p 10 |
| EP 1027883 | Comp I-VI, p 3 |
| EP 969004 | Ex 5, T 1, pp 6-8 |
| U.S. Pat. No. 5801244 | Ex 1-5, pp 6-7 |
| EP 832642 | Ex 22, T 3 pp, 10-15; T 4, p 16 |
| U.S. Pat. No. 5346691 (EP | Ex 40, p 7; T 5, p 8 |
| 570838) | |
| EP 517104 | Ex 1, T 1, pp 4-5; Ex 8, T 2, pp 6-8 |
| WO 200149686 | Ex 1-5, pp 16-21 |
| EP 944624 | Ex 1 + 2, pp 13-15 |
| EP 933376 | Ex 1-15, pp 10-21 |
| EP 863145 | Ex 1-11, pp 12-18 |
| EP 780382 | Ex 1-11, pp 5-7 |
| EP 626950 | All examples |
| EP 1081140 | Ex 1-9, pp 11-16 |
| WO 9217461 | Ex 1-22, pp 10-20 |
| WO 0168047 | Tables on pp 85-96 |
| EP 613893 | Ex 1-5 + 15, T 1, pp 6-8 |
| EP 1064922 | Compounds 1-34, pp 6-14 |
| EP 1028120 | Ex 1-5, pp 5-13 |
| EP 1008593 | Ex 1-8, pp 4-5 |
| EP 669323 | Ex 1-3, p 5 |
| EP 1108712 | 4,5-Dimorpholino-3-hydroxypyridazine |
| JP 2000319629 | CAS Regno. 80142-49-0, 137215-83-9, |
| | 307947-82-6 |
| EP 420707 B1 | Ex 3, p 13 (80142-49-0) |
| U.S. Pat. No. 5635343 | All examples |
| EP 1167358 | All examples |

(Abbreviations T: table, R: row, Comp: compound, Ex: compound(s) of patent example, p = page; pp = pages)

Synergistic effects can be observed when UV absorbers are used in combination with antioxidants. Examples of antioxidants that can be used are listed in WO 01/36396 (pages 11-18), U.S. Pat. No. 5,922,310 and U.S. Pat. No. 4,786,493.

Suitable cosmetic preparations may contain usually from 0.05 to 40% by weight, preferably from 0.1 to 20% by weight, based on the total weight of the composition, of one or more UV absorbers.

In a further embodiment of the present invention the UV absorbers are micronised, for example, by:
wet-grinding with a hard grinding medium, for example zirconium silicate and a protective surfactant or a protective polymer in water or in a suitable organic solvent;
spray-drying from a suitable solvent, for example aqueous suspensions or suspensions containing organic solvents, or true solutions in water, ethanol, dichloroethane, toluene or N-methylpyrrolidone etc.;
by the expansion according to the RESS process LRapid Expansion of Supercritical Solutions) of supercritical fluids (e.g. CO₂) in which the UV filter or filters is/are dissolved, or the expansion of fluid carbon dioxide together with a solution of one or more UV filters in a suitable organic solvent;
by reprecipitation from suitable solvents, including supercritical fluids (GASR process=Gas Anti-Solvent Recrystallisation/PCA process=Precipitation with Compressed Anti-solvents).

As grinding apparatus for the preparation of the micronised organic UV absorbers there may be used, for example, a jet mill, ball mill, vibratory mill or hammer mill, preferably a high-speed mixing mill. The grinding is preferably carried out with a grinding aid, for example an alkylated vinylpyrrolidone polymer, a vinylpyrrolidone/vinyl acetate copolymer, an acyl glutamate, an alkyl polyglucoside, ceteareth-25 or a phospholipid.

The micronised UV absorbers so obtained usually have an average particle size that is from 0.02 to 2 μm, preferably from 0.05 to 1.5 μm, and more especially from 0.1 to 1.0 μm.

The UV absorbers can also be used dry in powder form. For that purpose the UV absorbers are subjected to known grinding methods, such as vacuum atomization, countercurrent spray-drying etc. Such powders have a particle size of from 0.1 μm to 2 μm. To avoid the occurrence of agglomeration, the UV absorbers can be coated with a surface-active compound prior to the pulverisation process, for example with an anionic, non-ionic or amphoteric surfactant, e.g. a phospholipid or a known polymer, such as PVP, or an acrylate. The preparations according to the invention may further comprise antimicrobial agents. Preferred antimicrobial preservatives and antimicrobial actives used in formulations (in most cases the INCI name of the antimicrobial substances is mentioned): formaldehyde and paraformaldehyde, hydroxy biphenyls and its salts such as ortho-phenylphenol, zinc pyrithion, chlorobutanol, hydroxy benzoic acids and their salts and esters such as methyl paraben, ethyl paraben, propyl paraben, butyl paraben, dibromo hexamidine and its salts including isothionate (4,4′-hexamethylenedioxy-bis(3-bromo-benzamidine) and 4,4′-hexamethylenedioxy-bis(3-bromo-benzamidinium 2-hydroxyethanesulfonate), mercury, (aceto-O)phenyl (especially phenyl mercuric acetate) and Mercurate(2-),(orthoborate(3-)-O)phenyl, dihydrogene (especially phenyl mercuric borate), 1,3-bis(2-ethylhexyl)-hexahydro-5-methyl-5-pyrimidine (Hexetidin), 5-bromo-5-nitro-1,3-dioxan, 2-bromo-2-nitro-1,3-propandiol, 2,4-dichlorobenzyl alcohol, 3,4,4′ trichlorocarbanilide (Trichlorcarban), p-chloro-m-cresol, 2,4,4′-trichloro 2-hydroxy diphenylether (triclosan), 4,4′-dichloro 2-hydroxy diphenylether, 4-chloro-3,5-dimethylphenol (Chloroxylenol), imidazolidinyl urea, poly-(hexamethylene biguanide) hydrochloride, 2-phenoxy ethanol (phenoxyethanol), hexamethylene tetramine (Methenamine), 1-(3-chloroallyl)-3,5,7-triaza-1-azonia-adamantanchloride (Quaternium 15), 1-(4-chlorophenyoxy)-1-(1-imidazolyl)3,3-dimethyl-2-butanone (Climbazole), 1,3-bis(hydroxymethyl)-5,5-dimethyl-2,4-imidazolidinedione (DMDM hydantoin), benzyl alcohol, 1,2-dibromo-2,4-dicyano butane, 2,2′ methylene-bis(6-bromo-4-chloro phenol) (bromochlorophene), methylchloroisothiazolone, methylisothiazolone, octylisothiazolone, benzylisothiazolorie, 2-benzyl-4-chlorophenol (Chlorophenone), chloracetamide, chlorhexidine, chlorhexidine acetate, chlorhexidine gluconate, chlorhexidine hydrochloride, 1-phenoxy-propane-2-ol (phenoxyisopropanol), 4,4-dimethyl-1,3-oxazolidine (dimethyl oxazolidine), diazolidinyl urea, 4,4′-hexamethylenedioxybisbenzamidine and 4,4′-hexamethylenedioxybis(benzamidinium-2-hydroxyethanesulfonate), glutaraldehyde (1,5-pentanedial), 7-ethylbicyclooxazolidine, 3-(4-chlorophenoxy)-1,2-propandiol (chlorophenesin), phenylmethoxymethanol and ((phenylmethoxy)methoxy)-methanol (benzylhemiformal), N-alkyl(C12-C22)trimethyl ammoniumbromide and -chloride (cetrimonium bromide, cetrimonium chloride), benzyl-dimethyl-(4-(2-(4-(1,1,3,3-tetramethylbutyl)-phenoxy)-ethoxy)-ethyl)-ammoniumchloride (benzethonium chloride), Alkyl-(C8-C1 8)-dimethyl-benzylammonium chloride, -bromide and saccharinate (benzalkonium chloride, benzalkonium bromide, benzalkonium saccharinate), benzoic acid and its salts and esters, propionic acid and its salts, salicylic acid and its salt, sorbic acid and its salts, sodium iodiate, inorganic sulfites and bisulfites such as sodium sulfite, dehydroacetic acid, formic acid, mercurate(1-ethyl)2-mercaptobenzoate(2-)-O,S-,hydrogene (Thiomersal or Thiomerosal), 10-undecylenic acid and its salts, octopirox (piroctone olamine), sodium hydroxy methyl-aminoacetate (sodium hydroxymethylglycinate), 3-iodo-2-propynyl butylcarbamate, 10-undecylenic acid, and sulfur.

Combinations with natural antimicrobials or chemically modified natural substances with antimicrobial activities such as chitosans and chitosan derivatives, famesol, plant extracts such as clove oil, blue cypres oil etc. can be also used.

Typical formulation examples of shampoo/body wash formulations, which will provide hair and/or body cleansing products along with a glitter effect and a color flop from light to dark/dark to light or from color to color, when viewed from different angles in package and on skin and hair, are given below:

| | Typical Range | | | | |
|---|---|---|---|---|---|
| | Based on Activity | 1A | 2A | 3A | 4A |
| Water | qs to 100% | ¹⁾⁾ | ¹⁾ | ¹⁾ | ¹⁾ |
| Conditioning Agent/Cationic | 0-2% | | | | |
| Polyquaterium-7 | | — | 0.3 | 1.0 | 0.5 |
| Cationic Guar | | — | — | 0.5 | — |
| Cetrimonium Chloride | | 1.0 | — | — | — |
| Dimethicone Copolyol | | 1.0 | — | 2.0 | — |
| Primary/Co Surfactant | 8-20% | | | | |
| Disodium Monolaurethsulfosuccinate | | — | 10.0 | — | — |
| Sodium Cocoyl Sarcosinate | | — | — | — | 10.0 |
| Sodium Laureth Sulfate | | 25.0 | — | 30.0 | 20.0 |
| Ammonium Laureth Sulfate | | — | 10.0 | — | — |
| Secondary Surfactant | 0-15% | | | | |
| Sodium Methyl Cocoyl Taurate | | 3.0 | — | — | — |
| Cocamidopropyl Betaine | | — | — | 10.0 | — |
| PEG 20 Sorbitan Laurate | | — | 4.0 | — | — |
| Cocamide MEA | | 3.0 | — | 5.0 | — |
| Decyl Glucoside | | 11.0 | — | — | — |
| Disodium Cocoamphodiacetate | | — | 8.0 | — | 5.0 |
| Thickeners | | | | | |
| Hydroxyethylcellulose | 0-5% | 1.5 | 2.0 | — | 2.5 |
| Magnesium Aluminum Silicate | 0-5% | — | — | 1.0 | — |
| Pearlizing/Opacifying Agent | 0-10% | | | | |
| Glycol Stearate | | 2.0 | 1.0 | — | 2.0 |
| Ethylene Glycol Distearate | | — | 2.0 | — | — |
| Active Ingredient | 0-1% | | | | |
| Triclosan | | — | 0.1 | — | 0.1 |
| Viscosity Adjuster | 0-2% | | | | |
| Sodium Chloride | | ²⁾ | ²⁾ | ²⁾ | ²⁾ |
| Vitamins/Proteins | 0-2% | | | | |
| Hydrolyzed Soy Protein | | 0.8 | — | 1.0 | — |
| Tocopherol | | — | 0.1 | — | — |
| Panthenol | | — | 0.1 | 0.1 | 0.1 |
| Fragrance | 0-2% | 0.5 | 0.5 | 0.5 | 0.5 |
| Chelating Agent | <0.10% | | | | |
| Disodium EDTA | | 0.1 | 0.1 | — | — |
| Tetrasodium EDTA | | — | — | 0.1 | 0.1 |
| pH Adjuster | <0.50% | | | | |
| TEA | | qs to | qs to | qs to | qs to |
| | | 6.0-7.0 | 6.0-7.0 | 6.0-7.0 | 6.0-7.0 |
| Preservative | 0-1% | | | | |
| DMDM Hydantoin | | 1.0 | 1.0 | — | — |
| Phenoxyethanol and Methylparaben and | | — | — | 1.0 | 1.0 |
| Propylparaben and Butylparaben and | | | | | |
| Isobutylparaben | | | | | |
| Color | 0-20% | | | | |
| Gloss Pigments | | 3.0 | 2.0 | 2.0 | 2.5 |

¹⁾qs to 100%;
²⁾as needed

Typical formulation examples of hair gel formulations, which will provide hair styling/fixative products along with a glitter effect and a color flop from light to dark/dark to light or from color to color when viewed from different angles on hair and in package, are given below:

| | Typical Range | | | | |
|---|---|---|---|---|---|
| Ingredient | Based on Activity | 1A | 2A | 3A | 4A |
| Water | qs to 100% | ¹⁾⁾ | ¹⁾ | ¹⁾ | ¹⁾ |
| SD Alcohol 200 Proof | 0-6% | 0 | 0 | 6 | 0 |
| Hair Holding Conditioning Polymer | | | | | |
| PVP | 1-5% | 5.0 | 3.0 | 4.0 | — |
| PVP/VA Copolymer | 1-5% | — | 2.0 | — | 5.0 |
| Polyquaterium-11 | 1-5% | — | — | 1.0 | — |
| Gelling Agent/Thickener | 0.25-1% | 0.5 | 0.7 | 0.8 | — |
| Carbomer | | | | | |
| Steareth-10 Allyl Ether/Acrylates | 0.25-1% | 1.0 | — | — | 1.0 |
| Copolymer | | | | | |
| Neutralizer | | | | | |
| TEA, 99% | 0.5-1.5% | — | qs to | — | qs to |
| | | | 7.5 | | 7.5 |
| NaOH, 50% | 0.5-1.5% | qs to | — | qs to | — |
| | | 7.5 | | 7.5 | |
| Nonionic Surfactant | | | | | |
| Oleth-20 | 0.5-3% | 2.5 | 1.0 | 2.0 | — |
| Laureth-23 | 0.5-3% | — | 1.0 | — | 1.5 |
| Active Ingredient | | | | | |
| Triclosan | 0-1% | — | 0.1 | — | 0.1 |
| Viscosity Adjuster | | | | | |
| Sodium Chloride | 0-2% | ²⁾ | ²⁾ | ²⁾ | ²⁾ |
| Vitamins | | | | | |
| Tocopherol | 0-1% | 0.1 | 0.1 | — | — |
| Panthenol | 0-1% | — | 0.1 | 0.1 | 0.1 |
| Fragrance | 0-2% | 0.5 | 0.5 | 0.5 | 0.5 |
| Chelating Agent | | | | | |
| Disodium EDTA | <0.10% | 0.1 | 0.1 | — | — |
| Tetrasodium EDTA | <0.10% | — | — | 0.1 | 0.1 |
| pH Adjuster | | | | | |
| TEA | <0.50% | qs to | qs to | qs to | qs to |
| | | 6.0-7.0 | 6.0-7.0 | 6.0-7.0 | 6.0-7.0 |
| Preservative | | | | | |
| DMDM Hydantoin | 0-1% | 1.0 | 1.0 | — | — |
| Phenoxyethanol and Methylparaben and | 0-1% | — | — | 1.0 | 1.0 |
| Propylparaben and Butylparaben and | | | | | |
| Isobutylparaben | | | | | |
| Color | | | | | |
| Gloss Pigments | 0-20% | 5.0 | 7.0 | 2.0 | 2.5 |

¹⁾qs to 100%;
²⁾as needed

The cosmetic and personal care preparations and formulations according to the invention may also be used in the form of lotions, creams, candles or soaps that are composed of the base substances conventionally employed in the cosmetics industry and a pigment according to the invention.

Typical formulation examples of lotions/creams, which will provide skin moisturizing, conditioning, soothing benefits along with a glitter effect and color flop from light to dark/dark to light or from color to color, in package and on skin, are given below:

| | Typical | | | | | |
|---|---|---|---|---|---|---|
| | Range Based | | | | | |
| Ingredient | on Activity | 1A | 2A | 3A | 4A | 5A |
| Water | qs to 100% | ¹⁾⁾ | ¹⁾ | ¹⁾ | ¹⁾ | ¹⁾ |
| Humectant | 0-10% | | | | | |
| Glycerin | | 1.0 | 5.0 | 2.5 | — | 4.0 |
| Scleroglucan | | 2.5 | — | — | — | — |
| Sorbitol | | — | 1.0 | — | — | — |
| PEG 400 | | — | — | 1.0 | 1.5 | 2.0 |
| Thickening agent | 0-3% | | | | | |
| Carbomer | | — | — | — | — | 0.8 |
| Polyacrylamide and C₁₃₋₁₄Isoparaffin and | | — | — | — | 0.8 | — |
| Laureth-7 | | | | | | |
| Steareth-10 Allyl Ether/Acrylates | | 1.0 | — | — | — | — |
| Copolymer | | | | | | |
| Polyquaterium-37 and Propylene Glycol | | — | — | 1.5 | — | — |
| Dicaprylate Dicaprate and PPG-1 | | | | | | |
| Trideceth-6 | | | | | | |
| Sodium acrylates copolymer and mineral | | — | 1.0 | — | — | — |
| oil and PPG-1 Trideceth-6 | | | | | | |
| Surfactant/Emulsifiers/Solubilizers | 0-10% | | | | | |
| Glyceryl Stearate | | 4.0 | 3.0 | 1.0 | 1.0 | 1.5 |
| Steareth-2 | | — | — | — | 0.7 | — |
| PEG-100 Stearate | | 1.3 | 2.0 | — | 0.5 | |
| Sorbitan Sequioleate | | 1.0 | | | | |
| Cetyl Alcohol | | 1.2 | 2.0 | 1.0 | — | 1.0 |
| Cetearyl Alcohol | | 1.3 | — | — | — | 1.5 |
| Stearyl Alcohol | | — | — | — | 1.0 | — |
| Stearic Acid | | — | — | 2.5 | — | 3.0 |
| Behenic Acid | | — | — | — | 1.0 | — |
| Oils/Esters | 0-10% | | | | | |
| Caprylic/Capric Triglyceride | | — | 2.0 | 1.5 | — | 2.0 |
| Squalane | | — | 1.0 | — | — | 1.0 |
| Diethylhexyl Maleate | | 3.5 | — | — | — | — |
| Mineral Oil | | — | — | — | 2.0 | — |
| Isocetyl Stearate | | 1.5 | — | — | — | — |
| Polysorbate 60 | | 1.0 | — | — | — | — |
| Decyl Oleate | | — | — | 0.5 | 0.8 | 1.5 |
| Cetyl Palmitate | | — | 1.0 | 0.5 | — | 1.0 |
| Silicone | 0-5% | | | | | |
| Cyclopentasiloxane | | 0.5 | 1.0 | 1.0 | 4.0 | 2.0 |
| Dimethiconol | | 0.3 | — | — | — | — |
| Dimethicone | | — | — | — | — | 0.8 |
| Vitamins | 0-1% | | | | | |
| Tocopherol | | — | 0.1 | — | 0.1 | 0.1 |
| Panthenol | | 0.2 | — | 0.1 | — | 0.1 |
| Fragrance | 0-2% | 0.5 | 0.5 | 0.5 | 0.5 | 0.5 |
| Chelating Agent | | | | | | |
| Disodium EDTA | <0.10% | 0.1 | 0.1 | — | 0.1 | — |
| Tetrasodium EDTA | <0.10% | — | — | 0.1 | — | 0.1 |
| pH Adjuster | | | | | | |
| NaOH | <0.50% | qs 6.0 | — | qs 6.0 | qs 6.0 | — |
| | | to 6.5 | | to 6.5 | to 6.5 | |
| TEA | <0.50% | — | qs 6.0 | — | — | qs 6.0 |
| | | | to 6.5 | | | to 6.5 |
| Preservative | | | | | | |
| DMDM Hydantoin | 0-1% | — | — | — | 1.0 | — |
| Phenoxyethanol and Methylparaben and | 0-1% | 1.0 | 1.0 | 1.0 | — | 1.0 |
| Propylparaben and Butylparaben and | | | | | | |
| Isobutylparaben | | | | | | |
| Color | | | | | | |
| Gloss Pigment | 0-20% | 3.5 | 2.0 | 4.0 | 3.5 | 5.0 |

¹⁾qs to 100%

Typical formulation examples of candles, which will provide a glitter effect as well as a color flop from light to dark/dark to light or from color to color when viewed from various angles, lit or unlit, are given below:

| | Typical Range | | | | |
|---|---|---|---|---|---|
| Ingredient | Based on Activity | 1A | 2A | 3A | 4A |
| Proprietary Candle Wax | qs to 100% | ¹⁾⁾ | ¹⁾ | ¹⁾ | ¹⁾ |
| Base (Alene Candle) | | | | | |
| Solubilizer | 0-10% | | | | |
| Polysorbate-20 | | 1.0 | — | 7.0 | 8.0 |
| Polysorbate-80 | | — | 4.0 | — | — |
| Fragrance | 0-8% | 2.0 | 4.0 | 6.0 | 8.0 |
| Color | 0-40% | | | | |
| Gloss Pigments | | 10.0 | 20.0 | 30.0 | 40.0 |

¹⁾qs to 100%

Typical formulation examples of soaps, which will provide cleansing along with a glitter effect and color flop from light to dark/dark to light or from color to color when viewed from various angles, on soap bars, on the hands, face and body and in the package, are given below:

| | Typical Range | | | | |
|---|---|---|---|---|---|
| Ingredient | Based on Activity | 1A | 2A | 3A | 4A |
| Proprietary Soap Base | qs to 100% | ¹⁾⁾ | ¹⁾ | ¹⁾ | ¹⁾ |
| (Bradford Soap | | | | | |
| Company) | | | | | |
| Fragrance | 0-3% | 0.5 | 1.0 | 2.0 | 3.0 |
| Color | 0-40% | | | | |
| Gloss Pigments | | 10.0 | 20.0 | 30.0 | 40.0 |

¹⁾qs to 100%

The cosmetic and personal care preparations and formulations according to the invention are prepared in conventional manner, for example by mixing or stirring the components together, optionally with heating so that the mixtures melt.

The gloss pigment generally comprises

(a1) a core consisting of a substantially transparent or metallically reflecting material, and

(a2) at least one coating substantially consisting of one or more silicon oxides, the molar ratio of oxygen to silicon being on average from 0.03 to 0.95.

Such colored luster pigments and their preparation is described in EP-A-0 803 549, which is incorporated herein by reference.

The particles of the plane-parallel structures of the gloss pigment (flakes) generally have a length of from 1 μm to 5 mm, a width of from 1 μm to 2 mm, and a thickness of from 20 nm to 2 μm, and a ratio of length to thickness of at least 2:1, the particles having two substantially parallel faces, the distance between which is the shortest axis of the core.

The flakes are not of a uniform shape. Nevertheless, for purposes of brevity, the flakes will be referred to as having a “diameter.” The flakes have a high plane-parallelism and a defined thickness in the range of ±10%, especially ±5% of the average thickness. It is presently preferred that the diameter of the flakes be in a preferred range of about 1-60 μm with a more preferred range of about 5-40 μm. Thus, the aspect ratio of the flakes of the present invention is in a preferred range of about 2.5 to 625 with a more preferred range of about 50 to 250.

The term “silicon oxide layer, wherein the molar ratio of oxygen to silicon being on average from 0.03 to 0.95, i.e. SiOₓ with 0.03≦x≦0.95″ means that the molar ratio of oxygen to silicon at the average value of the silicon oxide layer is from 0.03 to 0.95. The composition of the silicon oxide layer can be determined by ESCA Lelectron spectroscopy for chemical analysis).

Accordingly, the term “SiO_{z} with 0.95<z<2.0″ means that the molar ratio of oxygen to silicon at the average value of the silicon oxide layer is from greater than 0.95 to 2.0. The composition of the silicon oxide layer can be determined by ESCA Lelectron spectroscopy for chemical analysis).

According to the present invention the term “aluminum” comprises aluminum and alloys of aluminum. Alloys of aluminum are, for example described in G. Wassermann in Ullmanns Enzyklopadie der Industriellen Chemie, 4. Auflage, Verlag Chemie, Weinheim, Band 7, S. 281 to 292. Especially suitable are the corrosion stable aluminum alloys described on page 10 to 12 of WO00/12634, which comprise besides of aluminum silicon, magnesium, manganese, copper, zinc, nickel, vanadium, lead, antimony, tin, cadmium, bismuth, titanium, Chromium andlor iron in amounts of less than 20% by weight, preferably less than 10% by weight.

The gloss pigment preferably has the following layer structure:

(a3) SiO_{z}, especially SiO₂,

(a2) at least one coating substantially consisting of one or more silicon oxides, the molar ratio of oxygen to silicon being on average from 0.03 to 0.95, preferably from 0.03 to 0.24,

(a1) a core consisting of a substantially transparent or metallically reflecting material, and

(a2) at least one coating substantially consisting of one or more silicon oxides, the molar ratio of oxygen to silicon being on average from 0.03 to 0.95, preferably from 0.03 to 0.24,

(a3) SiO_{z}, especially SiO₂, or

(a4) a coating consisting of any solid material, the composition of which differs from that of the coating (a3),

(a3) SiO_{z}, especially SiO₂,

(a2) at least one coating substantially consisting of one or more silicon oxides, the molar ratio of oxygen to silicon being on average from 0.03 to 0.95, preferably from 0.03 to 0.24,

(a1) a core consisting of a substantially transparent or metallically reflecting material, and

(a2) at least one coating substantially consisting of one or more silicon oxides, the molar ratio of oxygen to silicon being on average from 0.03 to 0.95, preferably from 0.03 to 0.24,

(a3) SiO_{z}, especially SiO₂,

(a4) a coating consisting of any solid material, the composition of which differs from that of the coating (a3), wherein 0.95<z≦2.0, especially 1.4≦z≦2.0.

If the core consists of a metallically reflecting material, that material is preferably selected from Ag, Al, Au, Cu, Cr, Ge, Mo, Ni, Si, Ti, Zn, alloys thereof, graphite, Fe₂O₃ and MoS₂. Special preference is given to Al.

If the core consists of a transparent material, the material is preferably selected from mica, SiO_{z}, in particular SiO₂ and SiO_{z}/TiO₂ mixtures. Special preference is given to SiO_{z}, wherein 0.95<z≦2.0, especially 1.4≦z≦2.0.

The material of coating (a4) is advantageously a metal oxide, such as, for example, TiO₂, ZrO₂, SiO, SiO₂, SnO₂, GeO₂, ZnO, Al₂O₃, V₂O₅, Fe₂O₃, Cr₂O₃, PbTiO₃ or CuO, or a mixture thereof.

The material of coating (a4) is preferably a dielectric material having a “high” refractive index, that is to say a refractive index greater than about 1.65, preferably greater than about 2.0, most preferred greater than about 2.2, which is applied to the entire surface of the silicon/silicon oxide substrate. Examples of such a dielectric material are zinc sulfide (ZnS), zinc oxide (ZnO), zirconium oxide (ZrO₂), titanium dioxide (TiO₂), carbon, indium oxide (In₂O₃), indium tin oxide (ITO), tantalum pentoxide (Ta₂O₅), chromium oxide (Cr₂O₃), cerium oxide (CeO₂), yttrium oxide (Y₂O₃), europium oxide (Eu₂O₃), iron oxides such as iron(II)/iron(III) oxide (Fe₃O₄) and iron(III) oxide (Fe₂O₃), hafnium nitride (HfN), hafnium carbide (HfC), hafnium oxide (HfO₂), lanthanum oxide (La₂O₃), magnesium oxide (MgO), neodymium oxide (Nd₂O₃), praseodymium oxide (Pr₆O₁₁), samarium oxide (Sm₂O₃), antimony trioxide (Sb₂O₃), silicon monoxides (SiO), selenium trioxide (Se₂O₃), tin oxide (SnO₂), tungsten trioxide (WO₃) or combinations thereof. The dielectric material is preferably metal oxide, it being possible for the metal oxide to be a single oxide or a mixture of oxides, with or without absorbing properties, for example TiO₂, ZrO₂, Fe₂O₃, Fe₃O₄, Cr₂O₃ or ZnO, with TiO₂ and ZrO₂ being especially preferred.

It is possible to obtain pigments that are more intense in colour and more transparent by applying, on top of the coating (a4), especially the TiO₂ layer, a metal oxide of low refractive index, such as SiO₂, Al₂O₃, AlOOH, B₂O₃ or a mixture thereof, preferably SiO₂ (WO093/08237).

Additional coatings may be applied in a manner known per se for the purpose of stabilisation with respect to weather and light.

The metal oxide layers are preferably applied by a wet-chemical method, in which context it is possible to employ the wet-chemical coating techniques developed for the preparation of pearl lustre pigments; techniques of this kind are described, for example, in DE-A-14 67 468, DE-A-19 59988, DE-A-20 09566, DE-A-22 14545, DE-A-22 15 191, DE-A-22 44298, DE-A-23 13 331, DE-A-25 22572, DE-A-31 37808, DE-A-31 37809, DE-A-31 51 343, DE-A-31 51 354, DE-A-31 51 355, DE-A-32 11 602 and DE-A-3235017, DE 1959988, WO 93/08237, and WO 98/53001, or else in further patent documents and other publications. For coating, the substrate particles are suspended in water, and one or more hydrolysable metal salts are added at a pH which is appropriate for hydrolysis and is chosen such that the metal oxides and/or metal oxide hydrates are precipitated directly onto the particles without any instances of secondary precipitation. The pH is kept constant usually by simultaneous metered addition of a base or alkali. The pigments are subsequently separated off, washed and dried and, if desired, calcined, the calcination temperature possibly being optimized in respect of the particular coating. If desired, following the application of individual coatings the pigments can be separated off, dried and, if desired, calcined before being resuspended for the application of further layers by precipitation (cf. U.S. Pat. No. 6,132,873).

In a preferred embodiment, the gloss pigment has the following layer structure: SiOₓ/SiO_{z}/SiOₓ, SiO_{z}/SiOₓ/SiO_{z}/SiOₓ/SiO_{z}, especially SiO₂/SiOₓ/SiO_{z}/SiOₓ/SiO₂, SiOₓ/Ai/SiOₓ, SiO_{z}/SiOₓ/Al/SiOₓ/SiO_{z}, especially SiO₂/SiOₓ/Al/SiOₓ/SiO₂, TiO₂/SiO_{z}/SiOₓ/SiO_{z}/SiOₓ/SiO_{z}/TiO₂, especially TiO₂/SiO₂/SiOₓ/SiO_{z}/SiOₓ/SiO₂/TiO₂ or TiO₂/SiO_{z}/SiOₓ/Al/SiOₓ/SiO_{z}/TiO₂, especially TiO₂/SiO₂/SiOₓ/Al/SiOₓ/SiO₂/TiO₂, wherein x is from 0.03 to 0.95 and 0.95<z≦2.0, especially 1.40<z≦2.0.

Gloss pigments comprising

(a1) a core consisting of SiO_{z} with 0.95<z≦2.0, in particular 1.4≦z≦2.0, and

(a2) at least one coating substantially consisting of one or more silicon oxides, the molar ratio of oxygen to silicon being on average from 0.03 to 0.95, especially gloss pigments having the following layer structure:

(a3) optionally a SiO₂coating,

(a2) a coating substantially consisting of one or more silicon oxides, the molar ratio of oxygen to silicon being on average from 0.03 to 0.95,

(a1) a core consisting of SiOz with 0.95<z≦2.0, in particular 1.40≦z≦2.0, and

(a2) a coating substantially consisting of one or more silicon oxides, the molar ratio of oxygen to silicon being on average from 0.03 to 0.95, and

(a3) optionally a SiO₂ coating, being novel and form a further aspect of the present invention. In this aspect of the present invention especially preferred gloss pigments having the following layer structure:

SiOₓ/SiO_{z}/SiOₓ, SiO_{z}/SiOₓ/SiO_{z}/SiOₓ/SiO_{z}, especially SiO₂/SiOₓ/SiO_{z}/SiOₓ/SiO₂, TiO₂/SiO_{z}/SiOₓ/SiO_{z}/SiOₓ/SiO_{z}/TiO₂, especially TiO₂/SiO₂/SiOₓ/SiO_{z}/SiOₓ/SiO₂/TiO₂, wherein z is from 0.95 to 2.0, especially from 1.40 to 2.00 and x is from 0.03 to 0.95, especially 0.05 to 0.50.

The gloss pigments are generally particles having a length of from 2 μm to 5 mm, a width of from 2 μm to 2 mm, and a thickness of from 20 nm to 1.5 μm, and a ratio of length to thickness of at least 2:1, the particles having a core of SiO_{z} having two substantially parallel faces, the distance between which is the shortest axis of the core, and an SiOₓ layer applied to those parallel faces, and, optionally, further layers. The further layers may be applied to the parallel faces or to the entire surface.

The core is a platelet having an average diameter of from 1 to 50 μm and a thickness of from 20 to 500 nm.

The thickness of the SiOₓ layer is generally from 5 to 200 nm, preferably from 5 to 100 nm.

The thickness of the SiO_{z} layer is generally from 1 to 200 nm, preferably from 2 to 100 nm.

The thickness of the TiO₂ layer is generally from 1 to 200 nm, preferably from 10 to 150 nm.

In addition, the present invention relates to gloss pigments comprising

(a1) a core consisting of a metallically reflecting material, and

(a2) at least one coating substantially consisting of one or more silicon oxides, the molar ratio of oxygen to silicon being on average from 0.03 to 0.24, especially gloss pigments having the following layer structure:

(a3) optionally a SiO_{z} coating with 0.95<z≦2.0, in particular 1.40≦z≦2.0,

(a2) a coating substantially consisting of one or more silicon oxides, the molar ratio of oxygen to silicon being on average from 0.03 to 0.24,

(a1) a core consisting of a metallically reflecting material, especially aluminum, and

(a2) a coating substantially consisting of one or more silicon oxides, the molar ratio of oxygen to silicon being on average from 0.03 to 0.24, and

(a3) optionally a SiOZ coating with 0.95<z≦2.0, in particular 1.40≦z≦2.0, being novel and form a further aspect of the present invention.

In this aspect of the present invention especially preferred gloss pigments having the following layer structure:

SiOₓ₁/Al/SiOₓ₁, SiO_{z}/SiOₓ₁/Al/SiOₓ₁/SiO_{z}, especially SiO₂/SiOₓ₁/Al/SiOₓ₁/SiO₂, and TiO₂/SiO_{z}/SiOₓ₁/Al/SiOₓ₁/SiOₓ₁/SiO_{z}/TiO₂, especially TiO₂/SiO₂/SiOₓ₁/Al/SiOₓ₁/SiO₂/TiO₂, wherein x1 is from 0.03 to 0.24 and 0.95<z≦2.0, preferably 1.4≦z≦2.0,

In addition, the present invention relates to gloss pigments having the following layer structure:

(a3) a SiO_{z} coating with 0.95<z≦1.95, in particular 1.40≦z≦1.80,

(a2) a coating substantially consisting of one or more silicon oxides, the molar ratio of oxygen to silicon being on average from 0.03 to 0.95,

(a1) a core consisting of a metallically reflecting material, especially aluminum, and

(a2) a coating substantially consisting of one or more silicon oxides, the molar ratio of oxygen to silicon being on average from 0.03 to 0.95, and

(a3) a SiO_{z} coating with 0.95<z≦1.95, in particular 1.40≦z≦1.80.

In this aspect of the present invention especially preferred gloss pigments having the following layer structure:

SiO_{z1}/SiOₓ/Al/SiOₓ/SiO_{z1}, or

TiO₂/SiO_{z1}/SiOₓ/Al/SiOₓ/SiO_{z1}/TiO₂, wherein

0.95<z1 ≦1.95, preferably 1.4<z1≦1.8 and x is from 0.03 to 0.95. z1 is preferably ≧1.0 and ≦1.8.

The core is a platelet having an average diameter of from 1 to 50 μm and consists preferably of aluminium. The thickness of the core depends on the used metal and is in case of aluminium of from 20 to 100 nm, especially 40 bis 60 nm.

The thickness of the SiOₓ layer is generally from 5 to 200 nm, preferably from 5 to 100 nm.

The thickness of the SiO_{z} layer is generally from 1 to 200 nm, preferably from 2 to 100 nm.

The thickness of the TiO₂ layer is generally from 1 to 200 nm, preferably from 10 to 150 nm.

The SiOₓ, SiO_{z}, SiO₂ and TiO₂ layers arranged mirror-symmetrically with respect to the core of Al, or SiO_{z} preferably have the same layer thickness. In a further embodiment of the invention, the substrate layer may be surrounded on both sides by metal oxides having different layer thicknesses.

Additional coatings may be applied in a manner known per se for the purpose of stabilisation with respect to weather and light.

For example, an SiO₂ protective layer can be applied on top of the titanium dioxide layer, for which the following method may be used: A soda waterglass solution is metered in to a suspension of the material being coated, which suspension has been heated to about 50-100° C., especially 70-80° C. The pH is maintained at from 4 to 10, preferably from 6.5 to 8.5, by simultaneously adding 10% hydrochloric acid. After addition of the waterglass solution, stirring is carried out for 30 minutes.

By using the gloss pigments according to the invention in cosmetics it is possible to obtain cosmetics whose colour changes in dependence upon the viewing angle (“flop effect”). In particular, the gloss pigments not having the TiO₂ coating, which consist only of silicon and oxygen, are, by virtue of the fact that they are free of heavy metals, outstandingly suitable for cosmetic applications.

The SiOₓ/SiO_{y} flakes, wherein 0.95<y≦1.8, preferably 1.0≦y≦1.5, are obtained by a method comprising the following steps:
a) vapour-deposition of a separating agent onto a (movable) carrier to produce a separating agent layer,
b) vapour-deposition of an SiOₓ layer onto the separating agent layer (0.03<x≦0.95),
c) vapour-deposition of an SiO_{y} layer (0.95<y≦1.8) onto the SiOₓ layer obtained in step b),
d) vapour-deposition of an SiOₓ layer (0.03<x≦0.95) onto the SiO_{y} layer obtained in step c),
e) dissolution of the separating agent layer in a solvent,
f) separation of the gloss pigment from the solvent,
   the SiO_{y} layer in step c) being vapour-deposited from a vaporiser containing a charge comprising a mixture of Si and SiO₂, SiO_{y} or a mixture thereof, and the SiOₓ layer being vapour-deposited from a vaporiser charged with silicon.

The method indicated above makes available gloss pigments which, compared to natural mica effect pigments as well as to effect pigments produced in wet procedures, have a high degree of plane parallelism and a defined thickness in the range of ±10%, preferably ±5%, of the average thickness.

The separating agent condensed onto the carrier may be a lacquer, a polymer, such as, for example, the thermoplastic polymers described in U.S. Pat. No. 6,398,999, an organic substance soluble in organic solvents or water and vaporisable in vacuo, such as anthracene, anthraquinone, acetamidophenol, acetylsalicylic acid, camphoric anhydride, benzimidazole, benzene-1,2,4-tricarboxylic acid, biphenyl-2,2-dicarboxylic acid, bis(4-hydroxyphenyl)sulfone, dihydroxyanthraquinone, hydantoin, 3-hydroxybenzoic acid, 8-hydroxyquinoline-5-sulfonic acid monohydrate, 4-hydroxycoumarin, 7-hydroxycoumarin, 3-hydroxynaphthalene-2-carboxylic acid, isophthalic acid, 4,4-methylene-bis-3-hydroxynaphthalene-2-carboxylic acid, naphthalene-1,8-dicarboxylic anhydride, phthalimide and its potassium salt, phenolphthalein, phenothiazine, saccharin and its salts, tetraphenylmethane, triphenylene, triphenylmethanol or a mixture of at least two of those substances. The separating agent is preferably an inorganic salt soluble in water and vaporisable in vacuo (see, for example, DE 198 44 357), such as sodium chloride, potassium chloride, lithium chloride, sodium fluoride, potassium fluoride, lithium fluoride, calcium fluoride, sodium aluminium fluoride and disodium tetraborate.

The SiO_{y} layer is obtained by heating a preferably stoichiometric mixture of fine silicon and quartz (SiO₂) powder in a vaporiser, which is described, for example, in DE 4342574 C1 and in U.S. Pat. No. 6,202,591, to more than 1300° C. under a high vacuum. The reaction product is silicon 10 monoxide gas, which under vacuum is directed directly onto the passing carrier, where it condenses as SiO. Non-stoichiometric mixtures may also be used. The vaporiser contains a charge comprising a mixture of Si and SiO₂, SiO_{y}, or a mixture thereof, the particle size of the substances that react with one another (Si and SiO₂) being advantageously less than 0.3 mm. The weight ratio of Si to SiO₂ is advantageously in the range from 0.15:1 to 0.75:1 (parts by weight); preferably, a stoichiometric mixture is present. SiO_{y} present in the vaporiser vaporises directly. Si and SiO₂ react at a temperature of more than 1300° C. to form silicon monoxide vapour.

In accordance with the invention, step e) is carried out at a pressure that is higher than the pressure in steps a) to d) and lower than atmospheric pressure.

The (movable) carrier preferably comprises one or more continuous metal belts, with or without a polymer coating, or one or more polyimide or polyethylene terephthalate belts. The (movable) carrier may furthermore comprise one or more discs, cylinders or other rotationally symmetrical bodies, which rotate about an axis.

The gloss pigments are separated from the solvent of the separating agent preferably by washing-out and subsequent filtration, sedimentation, centrifugation, decanting or evaporation. Furthermore, the gloss pigments may, after washing-out of the dissolved separating agent contained in the solvent, be frozen together with the solvent and subsequently subjected to a process of freeze-drying, whereupon the solvent is separated off as a result of sublimation below the triple point and the dry product remains behind in the form of individual plane-parallel structures.

The silicon suboxide condensed on the movable carrier starting from vaporised SiO corresponds to the formula SiO_{y} wherein 0.95<y≦1.8, preferably wherein 1.1≦y≦1.5, y values of less than 1 being obtained by means of an excess of silicon in the vaporiser material. Except under an ultra-high vacuum, in industrial vacuums of a few 10⁻² Pa vaporised SiO always condenses as SiO_{y} wherein 1≦y≦1.8, especially wherein 1.1≦y≦1.5, because high-vacuum apparatuses always contain, as a result of gas emission from surfaces, traces of water vapour which react with the readily reactive SiO at vaporisation temperature. The SiO_{y} layers can be converted into SiO_{y+a} layers by means of oxidative heat treatment, wherein 0.05≦a≦1.05.

If, under industrial vacuums of a few 10⁻² Pa, Si is vaporised instead of SiO, silicon oxides are obtained which have a less-than-equimolar oxygen content, that is to say SiOₓ wherein 0.03≦x≦0.95, especially 0.05≦x≦0.50, very especially 0.10≦x≦0.30, which have astonishingly high stability to oxidation along with a high refractive index, even in thin layers. Heating in the presence of oxygen at from 150 to 500° C., preferably from 175 to 300° C., unexpectedly results in a very thin, e.g. approximately 20 nm thick, superficial silicon dioxide layer, which represents a very convenient method of producing structures having the layer sequence SiO₂/SiOₓ/SiO_{z}/SiOₓ/SiO₂, wherein 0.95<z≦2.0, especially 1.40≦z≦2.0. If thicker silicon dioxide layers are desired, they may conveniently be produced, as described above, by means of vapour-deposition of SiO_{y} and oxidative heat treatment thereof.

In detail, a salt, for example NaCl, followed successively by the layers of SiOₓ and SiO_{y}, is vapour-deposited onto a carrier, which may be a continuous metal belt, passing by way of the vaporisers under a vacuum of <0.5 Pa, the vapour-deposited thicknesses of salt being about from 20 to 100 nm, preferably from 30 to 60 nm. On its further course, the belt-form carrier, which is closed to form a loop, runs through dynamic vacuum lock chambers of known mode of construction (cf. U.S. Pat. No. 6,270,840) into ae region of from 1 to 5×10⁴ Pa pressure, preferably from 600 to 10⁹ Pa pressure, and especially from 10³ to 5×10³ Pa pressure, where it is immersed in a dissolution bath. The temperature of the solvent, i.e. water in case of a salt, should be so selected that its vapour pressure is in the indicated pressure range. With mechanical assistance, the separating agent layer rapidly dissolves and the product layer breaks up into flakes, which are then present in the solvent in the form of a suspension. On its further course, the belt is dried and freed from any contaminants still adhering to it. It runs through a second group of dynamic vacuum lock chambers back into the vaporisation chamber, where the process of coating with separating agent and product layer is repeated.

The suspension then present in both cases, comprising product structures and solvent, and the separating agent dissolved therein, is then separated in a further operation in accordance with a known technique. For that purpose, the product structures are first concentrated in the liquid and rinsed several times with fresh solvent in order to wash out the dissolved separating agent. The product, in the form of a solid that is still wet, is then separated off by filtration, sedimentation, centrifugation, decanting or evaporation.

After drying, the product, i.e. the SiO_{y} layers, can be subjected to oxidative heat treatment. Known methods are available for that purpose. Air or some other oxygen-containing gas is passed through the plane-parallel structures, which are in the form of loose material or in a fluidised bed, for several hours at a temperature of more than 200° C., preferably more than 400° C. and especially from 500 to 1000° C. The product can then be brought to the desired particle size by means of grinding or air-sieving and delivered for further use.

Separating off the plane-parallel structures after washing-out at atmospheric pressure can be carried out under gentle conditions by freezing the suspension, which has been concentrated to a solids content of about 50%, and subjecting it in known manner to freeze-drying at about −10° C. and 50 Pa pressure. The dry substance remains behind as product, which can be subjected to the steps of further processing by means of coating or chemical conversion. Instead of using a continuous belt, it is possible to produce the product by carrying out the steps of vapour-deposition of separating agent and SiO, of dissolution, and of drying the carrier, in an apparatus having a rotary body, in accordance with DE-A-199 52 032. The rotary body may be one or more discs, a cylinder or any other rotationally symmetrical body.

The method described hereinbefore makes it possible to produce gloss pigments with a high production rate, having very good stability properties and being distinguished by a wide range of possible hues with good colour saturation and hiding power.

The gloss pigments produced in accordance with the method according to the invention especially have a high degree of colour purity and gloss and are highly shear-stable. The pigment platelets dissolved away from the carrier have, with respect to one another, substantially identical and reproducible optical properties, such as, for example, the same hue when viewed from a particular angle, because the thickness of the layers can be readily controlled.

The optional coating with TiO₂ can result in more intense colours and is preferably applied by precipitation by wet chemical means.

The titanium oxide layers are obtainable, for example, in analogy to a method described in DE-A-195 01 307, by producing the titanium oxide layer by controlled hydrolysis of one or more titanic acid esters, where appropriate in the presence of an organic solvent and a basic catalyst, by means of a sol-gel process. Suitable basic catalysts are, for example, amines, such as triethylamine, ethylenediamine, tributylamine, dimethylethanolamine and methoxypropylamine.

The organic solvent is a water-miscible organic solvent such as a C₁₋₄alcohol, especially isopropanol.

Suitable titanic acid esters are selected from the group comprising alkyl and aryl alcoholates, carboxylates, and carboxyl-radical- or alkyl-radical- or aryl-radical-substituted alkyl alcoholates or carboxylates of titanium. The use of tetraisopropyl titanate is preferred. In addition, acetylacetonates and acetoacetylacetonates of titanium, such as titanium acetylacetonate, may be used.

In accordance with an embodiment of the present invention, the method described in U.S. Pat. No. 3,553,001 is used for application of the titanium dioxide layers.

An aqueous titanium salt solution is slowly added to a suspension of the material being coated, which suspension has been heated to about 50-100° C., especially 70-80° C., and a substantially constant pH value of about from 0.5 to 5, especially about from 1.2 to 2.5, is maintained by simultaneously metering in a base such as, for example, aqueous ammonia solution or aqueous alkali metal hydroxide solution. As soon as the desired layer thickness of precipitated TiO₂ has been achieved, the addition of titanium salt solution and base is stopped.

This method, also referred to as a titration method, is distinguished by the fact that an excess of titanium salt is avoided. That is achieved by feeding in for hydrolysis, per unit time, only that amount which is necessary for even coating with the hydrated TiO₂ and which can be taken up per unit time by the available surface of the particles being coated. In principle, the anatase form of TiO₂ forms on the surface of the starting pigment. By adding small amounts of SnO₂, however, it is possible to force the rutile structure to be formed. For example, as described in WO 93/08237, tin dioxide can be deposited before titanium dioxide precipitation and the anatase form of TiO₂ can be converted to the rutile form of TiO₂ by calcinating at 800 to 900° C.

Where appropriate, an SiO₂ protective layer can be applied on top of the titanium dioxide layer, for which the following method may be used: A soda waterglass solution is metered in to a suspension of the material being coated, which suspension has been heated to about 50-100° C., especially 70-80° C. The pH is maintained at from 4 to 10, preferably from 6.5 to 8.5, by simultaneously adding 10% hydrochloric acid. After addition of the waterglass solution, stirring is carried out for 30 minutes.

It is possible to obtain pigments that are more intense in colour and more transparent by applying, on top of the TiO₂ layer, a metal oxide of low refractive index, such as SiO₂, Al₂O₃, AlOOH, B₂O₃ or a mixture thereof, preferably SiO₂, and applying a further TiO₂ layer on top of the latter layer.

It is furthermore possible to subject the finished pigment to subsequent coating or subsequent treatment which further increases the light, weather and chemical stability or which facilitates handling of the pigment, especially its incorporation into various media. For example, the procedures described in DE-A-22 15 191, DE-A-31 51 354, DE-A-32 35 017 or DE-A-33 34 598 are suitable as subsequent treatment or subsequent coating.

The particles of the gloss pigment according to the invention can, where appropriate, be integrated into a polymer, for example obtainable by emulsion polymerisation as known per se in many variants for toner particles, or by incorporation into a thermoplastic or a polymer dispersion or solution.

The gloss pigments according to the invention can be used for all customary purposes, for example for colouring polymers in the mass, surface coatings (including effect finishes, including those for the automotive sector) and printing inks, and also, for example, for applications in cosmetics. Such applications are known from reference works, for example “Industrial Organic Pigments” (W. Herbst and K. Hunger, VCH Verlagsgesellschaft mbH, Weinheim/New York, 2nd, completely revised edition, 1997).

The gloss pigments (effect pigments) according to the invention exhibit goniochromatic properties (“colour travel”, i.e. produce different colours depending on light incidence and viewing angle) and result in brilliant, highly saturated (lustrous) colours. They are accordingly very especially suitable for combination with conventional, transparent pigments, for example organic pigments such as, for example, diketopyrrolopyrroles, quinacridones, dioxazines, perylenes, isoindolinones etc., it being possible for the transparent pigment to have a similar colour to the effect pigment. Especially interesting combination effects are obtained, however, in analogy to, for example, EP 388 932 or EP 402 943, when the colour of the transparent pigment and that of the effect pigment are complementary.

The gloss pigments according to the invention can be used with excellent results for pigmenting high molecular weight organic material.

The high molecular weight organic material for the pigmenting of which the gloss pigments or pigment compositions according to the invention may be used may be of natural or synthetic origin. High molecular weight organic materials usually have molecular weights of about from 10³ to 10⁸ g/mol or even more. They may be, for example, natural resins, drying oils, rubber or casein, or natural substances derived therefrom, such as chlorinated rubber, oil-modified alkyd resins, viscose, cellulose ethers or esters, such as ethylcellulose, cellulose acetate, cellulose propionate, cellulose acetobutyrate or nitrocellulose, but especially totally synthetic organic polymers (thermosetting plastics and thermoplastics), as are obtained by polymerisation, polycondensation or polyaddition. From the class of the polymerisation resins there may be mentioned, especially, polyolefins, such as polyethylene, polypropylene or polyisobutylene, and also substituted polyolefins, such as polymerisation products of vinyl chloride, vinyl acetate, styrene, acrylonitrile, acrylic acid esters, methacrylic acid esters or butadiene, and also copolymerisation products of the said monomers, such as especially ABS or EVA.

From the series of the polyaddition resins and polycondensation resins there may be mentioned, for example, condensation products of formaldehyde with phenols, so-called phenoplasts, and condensation products of formaldehyde with urea, thiourea or melamine, so-called aminoplasts, and the polyesters used as surface-coating resins, either saturated, such as alkyd resins, or unsaturated, such as maleate resins; also linear polyesters and polyamides, polyurethanes or silicones.

The said high molecular weight compounds may be present singly or in mixtures, in the form of plastic masses or melts. They may also be present in the form of their monomers or in the polymerised state in dissolved form as film-formers or binders for surface coatings or printing inks, such as, for example, boiled linseed oil, nitrocellulose, alkyd resins, melamine resins and urea-formaldehyde resins or acrylic resins.

Depending on the intended purpose, it has proved advantageous to use the gloss pigments or gloss pigment compositions according to the invention as toners or in the form of preparations. Depending on the conditioning method or intended application, it may be advantageous to add certain amounts of texture-improving agents to the effect pigment before or after the conditioning process, provided that this has no adverse effect on use of the effect pigments for colouring high molecular weight organic materials, especially polyethylene. Suitable agents are, especially, fatty acids containing at least 18 carbon atoms, for example stearic or behenic acid, or amides or metal salts thereof, especially magnesium salts, and also plasticisers, waxes, resin acids, such as abietic acid, rosin soap, alkylphenols or aliphatic alcohols, such as stearyl alcohol, or aliphatic 1,2-dihydroxy compounds containing from 8 to 22 carbon atoms, such as 1,2-dodecanediol, and also modified colophonium maleate resins or fumaric acid colophonium resins. The texture-improving agents are added in amounts of preferably from 0.1 to 30% by weight, especially from 2 to 15% by weight, based on the end product.

The gloss pigments according to the invention can be added in any tinctorially effective amount to the high molecular weight organic material being pigmented. A pigmented composition comprising a high molecular weight organic material and from 0.01 to 80% by weight, preferably from 0.1 to 30% by weight, based on the high molecular weight organic material, of an effect pigment according to the invention is advantageous. Concentrations of from 1 to 20% by weight, especially of about 10% by weight, can often be used in practice. High concentrations, for example those above 30% by weight, are usually in the form of concentrates (“masterbatches”), which can be used as colorants for producing pigmented materials having a relatively low pigment content, the pigments according to the invention having an extraordinarily low viscosity in customary formulations so that they can still be processed well.

For the purpose of pigmenting organic materials, the effect pigments according to the invention may be used singly. It is, however, also possible, in order to achieve different hues or colour effects, to add any desired amounts of other colour-imparting constituents, such as white, coloured, black or effect pigments, to the high molecular weight organic substances in addition to the effect pigments according to the invention. When coloured pigments are used in admixture with the effect pigments according to the invention, the total amount is preferably from 0.1 to 10% by weight, based on the high molecular weight organic material. Especially high goniochromicity is provided by the preferred combination of an effect pigment according to the invention with a coloured pigment of another colour, especially of a complementary colour, with colorations made using the effect pigment and colorations made using the coloured pigment having, at a measurement angle of 10°, a difference in hue (ΔH*) of from 20 to 340, especially from 150 to 210.

Preferably, the gloss pigments according to the invention are combined with transparent coloured pigments, it being possible for the transparent coloured pigments to be present either in the same medium as the effect pigments according to the invention or in a neighbouring medium. An example of an arrangement in which the effect pigment and the coloured pigment are advantageously present in neighbouring media is a multi-layer effect surface coating.

The pigmenting of high molecular weight organic substances with the pigments according to the invention is carried out, for example, by admixing such a pigment, where appropriate in the form of a masterbatch, with the substrates using roll mills or mixing or grinding apparatuses. The pigmented material is then brought into the desired final form using methods known per se, such as calendaring, compression moulding, extrusion, coating, pouring or injection moulding. Any additives customary in the plastics industry, such as plasticisers, fillers or stabilisers, can be added to the polymer, in customary amounts, before or after incorporation of the pigment. In particular, in order to produce non-rigid shaped articles or to reduce their brittleness, it is desirable to add plasticisers, for example esters of phosphoric acid, phthalic acid or sebacic acid, to the high molecular weight compounds prior to shaping.

For pigmenting surface coatings and printing inks, the high molecular weight organic materials and the gloss pigments according to the invention, where appropriate together with customary additives such as, for example, fillers, other pigments, siccatives or plasticisers, are finely dispersed or dissolved in the same organic solvent or solvent mixture, it being possible for the individual components to be dissolved or dispersed separately or for a number of components to be dissolved or dispersed together, and only thereafter for all the components to be brought together.

Dispersing an effect pigment according to the invention in the high molecular weight organic material being pigmented, and processing a pigment composition according to the invention, are preferably carried out subject to conditions under which only relatively weak shear forces occur so that the effect pigment is not broken up into smaller portions.

The colorations obtained, for example in plastics, surface coatings or printing inks, especially in surface coatings or printing inks, more especially in surface coatings, are distinguished by excellent properties, especially by extremely high saturation, outstanding fastness properties and high goniochromicity.

When the high molecular weight material being pigmented is a surface coating, it is especially a speciality surface coating, very especially an automotive finish.

The following examples are for illustrative purposes only and are not to be construed to limit the scope of the instant invention in any manner whatsoever. Unless otherwise indicated, percentages and parts are percentages and parts by weight, respectively.

### EXAMPLE 1

A layer of about 50 nm of NaCl is vapour-deposited onto a metallic carrier in a vacuum chamber at a pressure of less than about 10⁻² Pa. Then, at the same pressure, the following materials are successively vapour-deposited: Si (at above 1850° C.), SiO (at from 1350 to 1550° C.) and Si (at above 1850° C.), as a result of which a film having the following layer structure is produced on the metal belt: SiOₓ/SiO/SiOₓ

The separating agent is then dissolved in water, whereupon flakes come away from the substrate. At atmospheric pressure, the resulting suspension is concentrated by filtration and rinsed several times with deionised water in order to remove Na⁺ and Cl⁻ ions that are present. That is followed by the steps of drying and, optionally, heating of the plane-parallel SiOₓ structures in the form of loose material at 200° C. for two hours in an oven through which air heated to 200° C. is passed. On heating of the platelets, an SiO₂ layer about 20 nm thick is formed on the surface, on the SiOₓ layer. After cooling, comminution and grading by air-sieving are carried out.

In accordance with the method described above, the products indicated in the following Table are obtained:

| | SiO_{0.2} | SiO₂ | SiO_{0.2} | | |
|---|---|---|---|---|---|
| Example | [nm] | [nm] | [nm] | Colour | Colour change |
| 1a | 45 | 160 | 45 | matt orange | matt orange to matt |
| | | | | | yellow-green |
| 1b | 45 | 240 | 45 | matt blue- | matt blue-green to |
| | | | | green | matt violet |
| 1c | 45 | 260 | 45 | glossy blue- | glossy blue-green to |
| | | | | green | glossy violet |
| 1d | 45 | 280 | 45 | glossy green | glossy green to |
| | | | | | violet |
| 1e | 45 | 440 | 45 | glossy | glossy yellow-green |
| | | | | yellow-green | to glossy green |

The pigments obtained in accordance with Example 1 exhibit a colour change when the viewing angle is changed.

A pigment sublimated onto a mirror-like steel belt, having the layer sequence SiOₓ (45 nm)/SiO_{y}(280 nm)/SiOₓ(45 nm), wherein x is 0.3 and y is from 0.95 to 1.2, exhibits the dependency of L/C/h values (standard illuminant D65; CIE31 colour coordinates) on viewing angle as Indicated in the following Table.

| Viewing angle | | | |
|---|---|---|---|
| (degrees) | L | C | h |
| 10 | 69 | 59.3 | 343 |
| 20 | 71.8 | 50.5 | 351 |
| 30 | 76.6 | 38.5 | 13 |
| 40 | 81 | 32.8 | 55.6 |
| 50 | 83.6 | 42.9 | 89.1 |
| 60 | 85.2 | 45 | 102.7 |

### EXAMPLE 2

Lipstick base having the following composition:

| Number | Substance | | Amount [%] |
|---|---|---|---|
| 1 | cera alba | | 11.4 |
| 2 | candelilla wax | | 8.1 |
| 3 | carnauba wax | | 3.8 |
| 4 | Lunacera M | | 6.0 |
| 5 | castor oil | | 38.8 |
| 6 | Controx KS | | 0.1 |
| 7 | aroma oil | | 1.0 |
| 8 | Amerlate P | | 2.5 |
| 9 | OH lan | | 1.6 |
| 10 | isopropyl palmitate | | 10.1 |
| 11 | Dow Corning 556 | | 2.8 |
| 12 | Dow Corning 1401 | | 3.3 |
| 13 | TiO₂ pigment | | 2.3 |
| 14 | pigment according to | | 8.2 |
| | Example 1a | | |
| | | Total | 100.0 |

Substances 8-10 are mixed together, and substances 13 and 14 are dispersed in the resulting mixture. The resulting paste is then passed several times through a three-roll apparatus. In the meantime, substances 1-6 are melted, stirred together until homogeneous, and then substances 7, 11 and 12 are stirred in. The two mixtures are then mixed together in the hot state until homogeneous distribution is achieved. The hot mass is then poured into a lipstick mould and allowed to cool. Lipsticks having an intense colour of outstanding light fastness and very good gloss, and exhibiting no bleeding, are obtained.

### EXAMPLE 3

Nail Varnish

A nail varnish has, for example, the following formulation [in %]:

| | |
|---|---|
| methyl acetate | 8.0 |
| ethyl acetate | 8.0 |
| propyl acetate | 12.0 |
| butyl acetate | 25.0 |
| polyester resin | 7.5 |
| nitrocellulose/isopropanol | 21.0 |
| toluenesulfonamide epoxy | 9.0 |
| resin | |
| camphor | 1.5 |
| dibutyl phthalate | 5.0 |
| stearalkonium hectorite | 1.0 |
| titanium dioxide | 0.6 |
| pigment according to | 1.2 |
| Example 1a | |
| bismuth oxychloride | 0.2 |

### EXAMPLE 4

Lipstick having the following composition:

| Phase | Ingredient | Tradename | parts |
|---|---|---|---|
| A | castor oil | Livopol CO¹⁾ | 36.70 |
| A | triethylhexanoin | Schercemol GTO²⁾ | 7.50 |
| A | triisostearyl trilinoleate | Schercemol TIST²⁾ | 15.00 |
| A | triisostearyl citrate | Schercemol TISC²⁾ | 17.50 |
| A | euphorbia cerifera wax | Candelilla Wax Prills³⁾ | 7.00 |
| A | copernicia cerifera wax | Carnuba Wax Prills³⁾ | 1.80 |
| A | ozokerite 77W | Ozokerite³⁾ | 1.80 |
| A | microcyrstalline wax 1275W³⁾ | | 3.50 |
| A | hydroxylated hanolin | Ritahydrox⁴⁾ | 1.00 |
| A | methylparaben | Nipagin M⁵⁾ | 0.20 |
| A | propylparaben | Nipasol M⁵⁾ | 0.10 |
| B | pigment according to | | 3.50 |
| | example 1a, 1b, 1c, 1d, or 1e | | |
| B | boron nitride | boron nitride powder⁶⁾ | 4.35 |
| B | water and tocopheryl acetate | Brookosome ACE⁷⁾ | 0.05 |
| | and retinayl palmitate and | | |
| | ascorbyl palmitate | | |
| | | total | |
| | | | 100.00 |

The ingredients of phase A are combined, heated between 90-105° C., and mixed until uniform. The ingredients of phase B are then added with stirring until homogenous. The temperature is maintained above 70° C. as the lipstick is poured into the mold.

### EXAMPLE 5

Talc free loose face powder having the following composition:

| Ingredient | Tradename | Parts |
|---|---|---|
| mica | Sericite PHN⁸⁾ | 90.95 |
| polymethyl methacrylate | Ganzpearl GM-0600⁸⁾ | 5.00 |
| synthetic wax and gluten protein | Microcase 110XF⁸⁾ | 2.00 |
| pigment according to example 1a, 1b, | | 1.50 |
| 1c, 1d, or 1e | | |
| methylparaben | Nipagin M⁵⁾ | 0.20 |
| propylparaben | Nipasol M⁵⁾ | 0.10 |
| imidazolidinyl urea | Germall 115⁹⁾ | 0.25 |
| | total | 100.00 |

All ingredients are combined in a blender and mixed well.

### EXAMPLE 7

Oil in water facial foundation having the following composition:

| Phase | Ingredient | Tradename | Parts |
|---|---|---|---|
| A | deionized water | water | 60.24 |
| A | 10% KOH solution | 10% KOH solution | 1.30 |
| A | PEG-12 dimethicone | DC 193 Surfactant¹⁰⁾ | 0.10 |
| A | pigment according to example | | 5.00 |
| | 1a, 1b, 1c, 1d, or 1e | | |
| A | talc | Talc¹¹⁾ | 0.72 |
| B | 1,3-butylene glycol | Jeechem BUGL¹²⁾ | 4.00 |
| C | 1,3-butylene glycol | Jeechem BUGL¹²⁾ | 2.00 |
| C | | Veegum Plus¹³⁾ | 0.12 |
| C | methylparaben | Nipagin M⁵⁾ | 0.02 |
| D | propylparaben | Nipasol M⁵⁾ | 0.10 |
| D | di-PPG-3 myristyl ether adipate | Cromollient DP3-A¹⁴⁾ | 14.00 |
| D | diethyl hexyl maleate | Pelemol DOM¹⁵⁾ | 4.00 |
| D | steareth-10 | Lipocol S-10¹⁾ | 2.00 |
| D | steareth-2 | Lipocol S-2¹⁾ | 0.50 |
| D | cetyl alcohol | Crodacol C-95 NF¹⁴⁾ | 0.62 |
| D | dicetyl phosphate and ceteth-10 | Crodapos CES¹⁴⁾ | 4.00 |
| | phosphate and ceteryl alcohol | | |
| D | propyl paraben | Nipasol M⁵⁾ | 0.10 |
| E | DMDM hydantoin | Mackstat DM¹⁶⁾ | 0.18 |
| | | total | 100.00 |

The ingredients of phase A are combined and heating to 80° C. is begun. The ingredients of phase B and C are added and homogenized for 1 h. In a separate beaker the ingredients of phase D are combined and heated to 80° C. After all the ingredients in phase D have become uniform they are slowly added to the main mixture while homogenization is continued. Upon complete addition of phase D, the formulation is homogenized for 15 min at 80° C., then cooled to 50° C. and phase E is added.

### EXAMPLE 8

Powder Eye Shadow having the following composition:

| Ingredient | Tradename | Parts |
|---|---|---|
| mica | Sericite PHN⁸⁾ | 76.20 |
| zinc stearate | Zinc Stearate¹⁶⁾ | 5.00 |
| titanium oxide | Titanium Oxide 3228¹⁷⁾ | 2.00 |
| pigment according to example 1a, 1b, | | 6.00 |
| 1c, 1d, or 1e | | |
| methylparaben | Nipagin M⁶⁾ | 0.20 |
| propylparaben | Nipasol M⁶⁾ | 0.10 |
| calcium aluminum borosilicate | Luxsil⁸⁾ | 5.00 |
| PEG-4 diheptanoate | Liponate 2-DH¹⁾ | 5.50 |
| | total | 100.00 |

The ingredients are combined and mixed well, heated to 100° C. and pressed at 2000 psi.

### EXAMPLE 9

Nail Enamel having the following composition:

| Phase | Ingredient | Tradename | Parts |
|---|---|---|---|
| A | butyl acetate and toluene and | Suspending Lacquer | 80.00 |
| | nitrocellulose and tosylamide/ | SLF-2¹⁸⁾ | |
| | formaldehyde resin and isopropyl | | |
| | alcohol and dibutyl phthalate and | | |
| | ethyl acetate and camphor and n- | | |
| | butyl alcohol and silica and | | |
| | quaterinium-18 hectorite | | |
| A | butyl acetate (and) bismuth | Biju Ultra UXD¹⁸⁾ | 2.00 |
| | oxychloride (and nitrocellulose | | |
| | (and) isopropyl alcohol (and) | | |
| | stearalkonium hectorite | | |
| A | mica (and) titanium dioxide | Flamenco Ultra | 1.00 |
| | | Sparkle 4500¹⁸⁾ | |
| A | pigment according to example 1a, | | 2.50 |
| | 1b, 1c, 1d, or 1e | | |
| A | dimethicone | Dow Corning 200¹⁰⁾ | 1.00 |
| A | tosylamide/epoxy resin | Lustrabrite S-70¹⁹⁾ | 4.00 |
| B | butyl acetate | | 1.17 |
| B | ethyl acetate | | 0.42 |
| B | toluene | | 1.66 |
| | | total | 100 |

The ingredients of phase A are combined and mixed until uniform. The ingredients of phase B are combined in a separate vessel and mixed until uniform. Phase B is added to phase A with stirring until uniform.

### EXAMPLE 10

Lip Gloss having the following composition:

| Phase | Ingredient | Tradename | Parts |
|---|---|---|---|
| A | pigment according to example 1a, | | 5.15 |
| | 1b, 1c, 1d, or 1e | | |
| B | C₂₄₋₃₀alcohol | Performacol 425²⁰⁾ | 1.75 |
| B | copernica cerifera | Carnuba Wax³⁾ | 1.70 |
| B | microcrystalline wax | Microwax 1275²¹⁾ | 4.00 |
| B | triisostearyl polyglycerol-3 dimer | Schercemol PTID²⁾ | 43.30 |
| | dilinoleate | | |
| B | triisostearyl citrate | Schercemol TISC²⁾ | 38.40 |
| B | PE/PVA copolymer soy glycerides | Enviropur 301²²⁾ | 5.00 |
| B | methylparaben | Methylparaben²³⁾ | 0.20 |
| B | propylparaben | Propylparaben²³⁾ | 0.10 |
| B | tocopherol | Tocopherol²⁴⁾ | 0.10 |
| C | flavor | Flavor²⁵⁾ | 0.30 |
| | | | 100.00 |

The ingredients of phase B are combined, heated between 85-87° C., and mixed until uniform. The ingredients of phase A are then added with stirring until homogenous. The temperature is decreased to 70-72° C. and the ingredients of phase C are added.

### EXAMPLE 11

Pressed powder having the following composition:

| Phase | Ingredient | Tradename | Parts |
|---|---|---|---|
| A | talc | Rose Talc⁸⁾ | 80.85 |
| A | zinc stearate | Zinc Stearate¹⁶⁾ | 3.00 |
| A | propylparaben | Propylparaben²³⁾ | 0.25 |
| A | methylparaben | Methylparaben²³⁾ | 0.20 |
| A | imidazolidinyl urea | Unicide¹⁾ | 0.10 |
| A | pigment according to example 1a, | | 0.60 |
| | 1b, 1c, 1d, or 1e | | |
| A | mica and boron nitride | Lipomic 601 BN¹⁾ | 3.00 |
| A | polyamide-12 triazaminostilbene- | LipoLight OAP/C¹⁾ | 10.00 |
| | disulfonate and polyoxymethylene | | |
| | urea | | |
| B | PEG-4 diheptanoate | Liponate 2-DH¹⁾ | 2.00 |
| | | | 100.00 |

The ingredients of phase A are mixed and the ingredients of phase B are added slowly with mixing.

### EXAMPLE 12

Cream to Powder Blush having the following composition:

| Phase | Ingredient | Tradename | Parts |
|---|---|---|---|
| A | didecene | Silkflo 362 NF¹⁾ | 22.40 |
| A | PEG-4 diheptanoate | Liponate 2DH¹⁾ | 5.00 |
| A | isopropyl palmitate | Protachem IPP²³⁾ | 6.50 |
| A | pentaerythrityl tetrastearate | Crodamol PETS¹⁴⁾ | 2.50 |
| A | pentaethrityl tetrabehenate | Liponate PB-4¹⁾ | 1.00 |
| A | copernicia cerifera | Carnuba Wax³⁾ | 5.00 |
| A | soy lecithin | Lipo Lecithin¹⁾ | 0.50 |
| B | titanium dioxide, oil dispersion | Titanium Dioxide¹⁷⁾ | 9.00 |
| B | pigment according to example | | 10.00 |
| | 1a, 1b, 1c, 1d, or 1e | | |
| B | kaolin | Colliodal Kaolin NF¹⁷⁾ | 12.30 |
| B | talc | Rose Talc⁸⁾ | 17.00 |
| B | nylon 6-12 | Orgasol 4000¹⁾ | 6.00 |
| B | propylparaben | Propylparaben²³⁾ | 0.30 |
| B | mica and silica | SM-2000⁸⁾ | 2.00 |
| B | black iron oxide | Black Iron Oxide²⁶⁾ | 0.50 |
| B | mica and boron nitride | Lipomic 601 BN¹⁾ | 2.00 |
| | | total | 100.00 |

The ingredients of phase A are mixed in a homogenizer and heated to 70 to 75° C. The ingredients of phase B are combined and mixed until uniform. Phase B is added to phase A and the temperature is maintained at 70 to 75° C. while mixing for 30 minutes. The mixture is poured into a container.

### EXAMPLE 13

Water resistant mascara having the following composition:

| Phase | Ingredient | Trade Name | Parts |
|---|---|---|---|
| A | water | | 51.20 |
| A | PEG-8 | Prochem 400²³⁾ | 1.50 |
| A | xanthan gum | Keltrol CG²⁸⁾ | 0.50 |
| A | methylparaben | Methylparaben²³⁾ | 0.20 |
| A | imidazoldinyl urea | Unicide U-13¹⁾ | 0.10 |
| A | tetrahydroxypropyl | Neutrol TE²⁷⁾ | 1.30 |
| | ethylenediamine | | |
| B | copernicia cerifera | Carnuba Wax³⁾ | 9.00 |
| B | cera alba | Bees Wax³⁾ | 4.50 |
| B | isoeiconsane | Fancol IE | 4.50 |
| B | polyisobutene | Permethyl 104A⁸⁾ | 4.50 |
| B | stearic acid | Emersol 132²⁹⁾ | 6.00 |
| B | glyceryl stearate | Lipo GMS 450¹⁾ | 1.00 |
| B | propylparaben | Propylparaben²³⁾ | 0.20 |
| B | benzyl laurate | Luvitol BL²⁷⁾ | 2.00 |
| C | black iron oxide²⁶⁾ | | 3.00 |
| C | pigment according to example | | 2.00 |
| | 1a, 1b, 1c, 1d, or 1e | | |
| D | PVP/VA copolymer | Luviskol VA 64W²⁷⁾ | 8.50 |
| | | | 100.00 |

The ingredients of phase A and B are mixed separately and heated to 85° C. The temperature is maintained and phase B is added to phase A and the mixture is homogenized until uniform. The ingredients of phase C are added to the mixture of phase A and B and mixed until uniform. Mixing is continued and the ingredients of phase D are added.
Supplier:
¹⁾Lipo Chemicals, Inc.;
²⁾Scher Chemicals, Inc.;
³⁾Ross Wax;
⁴⁾Rita;
⁵⁾Clariant AG;
⁶⁾Ciba SC;
⁷⁾Brooks Industries, Inc.;
⁸⁾Presperse, Inc.;
⁹⁾International Specialty Products (ISP);
¹⁰⁾Dow Corning;
¹¹⁾Whittaker, Clark and Daniels, Inc;
¹²⁾Jeen International,
¹³⁾R.T. Vanderbilt Co., Inc.;
¹⁴⁾Croda International;
¹⁵⁾Phoenix Chemical Inc.,
¹⁶⁾McIntyre Group Ltd.;
¹⁶⁾Witco Corp.;
¹⁷⁾Whittaker, Clark & Daniels;
¹⁸⁾Engelhard Corp.;
¹⁹⁾Telechemische, Inc.;
²⁰⁾New Phase Technology;
²¹⁾Strahl and Pitsch Inc.;
²²⁾React Inc;
²³⁾Protameen Chemicals;
²⁴⁾Roche Vitamins;
²⁵⁾Atlanta Fragrance;
²⁶⁾Warner Jenkinson Cosmetic Colors;
²⁷⁾BASF AG;
²⁸⁾CP Kelco;
²⁹⁾Cognis AG.

1. A cosmetic and personal care preparation comprising
(a) from 0.0001 to 90% by weight of a gloss pigment comprising
(a1) a core consisting of a substantially transparent or metallically reflecting material, and
(a2) at least one coating substantially consisting of one or more silicon oxides, the molar ratio of oxygen to silicon being on average from 0.03 to 0.95, and
(b) from 10 to 99.9999% of a cosmetically suitable carrier material, based on the total weight of the cosmetic preparation or formulation.

2. A preparation according to claim 1, wherein the core consists of a metallically reflecting material selected from the group consisting of Ag, Al, Au, Cu, Cr, Ge, Mo, Ni, Si, Ti, Zn, or alloys thereof, graphite, Fe₂O₃ and MoS₂.

3. A preparation according to claim 1, wherein the core consists of a transparent material selected from the group consisting of mica and SiO_{z}, wherein 0.95<z<2.0.

4. A preparation according to claim 1, wherein the gloss pigment has a layer structure, said layer structure being: SiOₓ/SiO_{z}/SiOₓ, SiO_{z}/SiOₓ/SiO_{z}/SiOₓ/SiO_{z}, SiOₓ/Al/SiO_{z}, SiO_{z}/SiOₓ/Al/SiOₓ/SiO_{z}, TiO₂/SiO_{z}/SiOₓ/SiO_{z}/SiOₓ/SiO_{z}/TiO₂ or TiO₂/SiO_{z}/SiOₓ/Al/SiOₓ/SiO_{z}/TiO₂, wherein x is from 0.03 to 0.95 and 0.95<z<2.0.

5. A preparation according to claim 4, wherein the gloss pigment has the following layer structure: SiO₂/SiOₓ/SiO_{z}/SiOₓ/SiO₂ or TiO₂/SiO₂/SiOₓ/SiO_{z}/SiO₂/SiO₂/TiO₂, wherein x is from 0.03 to 0.90, and 0.95<z≦2.0.

6. A pigment comprising
(a1) a core consisting of SiO_{z} with 0.95<z≦2.0, and
(a2) at least one coating substantially consisting of one or more silicon oxides, the molar ratio of oxygen to silicon being on average from 0.03 to 0.95.

7. A pigment according to claim 6, wherein the pigment has a layer structure, said layer structure being:
(a3) optionally a SiO₂ coating,
(a2) a coating substantially consisting of one or more silicon oxides, the molar ratio of oxygen to silicon being on average from 0.03 to 0.95,
(a1) a core consisting of SiO_{z} with 0.95<z≦2.0, and
(a2) a coating substantially consisting of one or more silicon oxides, the molar ratio of oxygen to silicon being on average from 0.03 to 0.95, and
(a3) optionally a SiO₂ coating.

8. A pigment according to claim 7 having the following layer structure:
SiOₓ/SiO_{z}/SiOₓ,
SiO_{z}/SiOₓ/SiO_{z}/SiOₓ/SiO_{z} or
TiO₂/SiO_{z}/SiOₓ/SiO_{z}/SiOₓ/SiO_{z}/TiO₂,
wherein x is from 0.03 to 0.95, and 0.95<z≦2.0.

9. A pigment comprising
(a) a core consisting of a metallically reflecting material, and
(b) at least one coating substantially consisting of one or more silicon oxides, the molar ratio of oxygen to silicon being on average from 0.03 to 0.24.

10. A pigment according to claim 9 having a layer structure, said layer structure being:
SiO_{z}/SiOₓ₁/Al/SiOₓ₁SiO_{z}, or
TiO₂/SiO_{z}/SiOₓ₁/Al/SiOₓ₁/SiO_{z}/TiO₂,
wherein z is from 0.95 to 2.0, and x is from 0.03 to 0.24.

11. A pigment according to claim 9 having a layer structure, said layer structure being:
(a3) a SiO_{z} coating with 0.95<z≦1.95,
(a2) a coating substantially consisting of one or more silicon oxides, the molar ratio of oxygen to silicon being on average from 0.03 to 0.95,
(a1) a core consisting of a metallically reflecting material, and
(a2) a coating substantially consisting of one or more silicon oxides, the molar ratio of oxygen to silicon being on average from 0.03 to 0.95, and
(a3) a SiO_{z} coating with 0.95<z≦1.95.

12. A pigment according to claim 11, having the following layer structure:
SiO_{z1}/SiOₓ/Al/SiOₓ/SiO_{z1}, or
TiO₂/SiO_{z1}/SiOₓ/Al/SiOₓ/SiO_{z1}/TiO₂, wherein 0.95<z1≦1.95, and x is from 0.03 to 0.95.

13. A composition comprising a high molecular weight organic material and from 0.01 to 80% by weight, based on the high molecular weight organic material, of a pigment according to claim 6.

14. A preparation according to claim 1, wherein the core consists of a transparent material selected from the group consisting of SiO₂ and SiO_{z}/TiO₂ mixtures.

15. A preparation according to claim 14, wherein the core consists of SiO_{z}/TiO₂ mixtures, wherein 0.95<z≦2.0.

16. A pigment according to claim 8 wherein x is from 0.05 to 0.50.

17. A pigment according to claim 11 wherein the layer (a1) is aluminium.

18. A composition comprising a high molecular weight organic material and from 0.01 to 80% by weight, based on the high molecular weight organic material, of a pigment according to claim 9.
